(19) United States
(12) Patent Application Publication
(10) Pub. No.: US 2023/0234850 A1
(43) Pub. Date: Jul. 27, 2023

(54) ADJUVANTED VACCINES

(71) Applicant: Merck Sharp & Dohme LLC, Rahway, NJ (US)

(72) Inventors: Akhilesh Bhambhani, Doylestown, PA (US); Muneeswara Babu Medi, Chalfont, PA (US); Maya Salnikova, Doylestown, PA (US); William James Smith, Harleysville, PA (US); David S. Thiriot, Harleysville, PA (US)

(73) Assignee: Merck Sharp & Dohme LLC, Rahway, NJ (US)

(21) Appl. No.: 18/172,695

(22) Filed: Feb. 22, 2023

Related U.S. Application Data
(62) Division of application No. 16/759,167, filed on Apr. 24, 2020, now abandoned.
(60) Provisional application No. 62/576,819, filed on Oct. 25, 2017.

(51) Int. Cl. C01B 25/36; A61K 47/52; A61K 39/39

(52) U.S. Cl. CPC C01B 25/36; A61K 47/52; A61K 39/39; A61K 2039/55505

(57) ABSTRACT

Vaccine formulations are described comprising physically separated, lyophilized antigens and adjuvant components, which may be in lyoparticle form, as well as methods of using and making such formulations. Reconstituted formulations are also described.

### BACKGROUND OF THE INVENTION

### Field of the Invention

The present invention provides vaccine formulations, which are useful for vaccination of a subject.

### Description of Related Art

Typically, aluminum adjuvants (alone or with vaccine antigens) in liquid formulations are damaged by freezing. While there have recently been some formulations described which can prevent or minimize freezing damage to aluminum adjuvants, methods and compositions that prevent adjuvant and vaccine freezing damage would be of significant value. In addition, methods and compositions that increase vaccine thermostability, and enhance flexibility in manufacturing, packaging, storage and use of the vaccine would be of significant value.

Furthermore, there are cases in which for example the combination of vaccine components for a prolonged duration results in loss of potency or immunogenicity, whereas the combination for a short period of time is not damaging and potentially advantageous for the vaccine. Advances in all these areas could be obtained by application of the present invention.

### BRIEF SUMMARY OF THE INVENTION

The present invention provides methods for preparing aluminum adjuvants and/or aluminum adjuvanted vaccines in final containers or delivery devices. In an embodiment of the invention, the aluminum adjuvant comprises aluminum phosphate, amorphous aluminum hydroxyphosphate and/or aluminum hydroxide (e.g., Merck aluminum phosphate adjuvant (MAPA) or Merck aluminum adjuvant (MAA)).

In an embodiment, the present invention provides a dried aluminum adjuvant that comprises the product of a reaction between an aluminum compound and a phosphate compound under basic conditions. In particular embodiments, the adjuvant is a lyobead and in further embodiments, the product is aluminum phosphate, amorphous aluminum hydroxyphosphate, amorphous aluminum hydroxyphosphate sulfate, or aluminum hydroxide.

The present invention provides an article of manufacture comprising components

(1) the dried aluminum adjuvant of any one of claims 1-3;

(2) a dried antigen or (3) antigen dissolved in water and, optionally,

(4) water,

wherein component (1), component (2) or (3), and component (4), if present, are each physically separated from each other and co-packaged.

In particular embodiments, one more of components (1), (2) or (3), and (4), if present, further comprises a salt, a buffer, a preservative, a surfactant and/or a sugar.

In particular embodiments, the salt is NaCl, the buffer is histidine, the surfactant is polysorbate, the preservative is m-cresol or phenol, and the sugar is Trehalose or sucrose.

In particular embodiments, components (1) and/or (2) are provided as lyobeads.

In particular embodiments, components (1) and (2) are each provided as lyobeads and the article of manufacture comprises a first container comprising a mixture of the component (1) and component (2) lyobeads and optionally, a second container comprising component (4), if present.

The present invention further provides a method for making a vaccine comprising combining the above components.

The present invention further provides an article of manufacture comprising (a) components

(1) a dried aluminum salt;

(2) a dried phosphate; and optionally

(3) water or (4) antigen dissolved in water,

wherein component (1), component (2), and components (3) or (4), if present, are physically separated and co-packaged; or, (b) components

(1) a dried aluminum salt;

(2) a dried phosphate;

(3) optionally water; and

(5) dried antigen,

wherein component (1), component (2), and component (3), if present, and component (5) are physically separated and co-packaged.

In particular embodiments of (a) component (3) or (4) is not present.

In particular embodiments of (a) one or more of component (1), component (2), and component (3) or (4), if present, further comprises a salt, a preservative, a buffer, a surfactant, and/or sugar; or of (b) one or more of component (1), component (2), component (5), and component (3), if present, further comprises a salt, a preservative, a buffer, a surfactant, and/or sugar.

In particular embodiments, the salt is NaCl, the buffer is histidine, the surfactant is polysorbate, and the sugar is sucrose, Trehalose, or combination thereof.

In particular embodiments, components (1), (2), and (5) are provided as lyobeads.

In particular embodiments of (a) components (1) and (2) are each provided as lyobeads and the article of manufacture comprises a first container comprising a mixture of components (1) and (2) lyobeads and optionally a second container comprising component (3) or (4), if present; of (b) components (1), (2), and (5) are each provided as lyobeads and the article of manufacture comprises (i) a first container comprising a mixture of components (1), (2), and (5) lyobeads and optionally a second container comprising component (3), if present; or, (ii) a first container comprising a mixture of components (1) and (2) lyobeads, optionally a second container comprising component (3), if present, and a third container comprising component (5) lyobeads.

In a further embodiment of the above article of manufacture, the antigen is:

a protein;

a toxin or a toxoid;

a polysaccharide;

one or more pneumococcal polysaccharide-protein conjugates;

a 33-21-valent mixture of pneumococcal polysaccharide-protein conjugates, 21-15-valent mixture of pneumococcal polysaccharide-protein conjugates, 15-7-valent mixture of pneumococcal polysaccharide-protein conjugates, or monovalent pneumococcal polysaccharide-protein conjugate;

a 33-valent mixture of pneumococcal polysaccharide-protein conjugates;

a 21-valent mixture of pneumococcal polysaccharide-protein conjugates;

a 15-valent mixture of pneumococcal polysaccharide-protein conjugates;

a 1-valent serotype 3 pneumococcal polysaccharide-protein conjugate; or

a 1-valent serotype 19A pneumococcal polysaccharide-protein conjugate.

In a further embodiment, the protein that is conjugated to a pneumococcal polysaccharide is a carrier protein selected from CRM197, diphtheria toxin fragment B (DTFB), DTFB C8, Diphtheria toxoid (DT), tetanus toxoid (TT), fragment C of TT, pertussis toxoid, cholera toxoid, meningococcal outer membrane protein complex (OMPC), E. coli LT, E. coli ST, exotoxin A from Pseudomonas aeruginosa, Protein D from Non-Typeable Haemophilus influenza, and combinations thereof.

The present invention further provides a method for making a vaccine comprising combining the components in the article of manufacture as disclosed previously.

The present invention further provides article of manufacture comprising components

(1) an antigen, which is dried (i) or in solution (ii);

(2) a dried aluminum-containing and/or calcium-containing reagent; and

(3) a dried phosphate reagent and, optionally, a base;

wherein each of components (1), (2) and (3) are physically separated from the other components and co-packaged; and

wherein said aluminum-containing and/or calcium-containing reagent and said phosphate reagent and, optionally, the base, react when reconstituted with water to form an adjuvant.

In a further embodiment of the article of manufacture, component (1)(i), component (2), and component (3) are each provided as lyobeads.

In a further embodiment of the article of manufacture, component (1)(i), component (2), and component (3) are each provided as lyobeads and the article of manufacture comprises a first container comprising a mixture of the component (1)(i), component (2), and component (3) lyobeads, and optionally, a second container comprising water.

The present invention further provides a method for making a vaccine comprising combining components (1)-(3) of the article of manufacture of above.

The present invention further provides article of manufacture comprising:

(1) a solution comprising an aluminum salt and a phosphate with a pH of about 5 or less and which is substantially clear; and

(2) a solution comprising base sufficient to cause a reaction and precipitation when added to the solution of (1), and an antigen; or a dried composition comprising a base sufficient to cause a reaction and precipitation when added to the solution of (1), and an antigen;

wherein each of (1) and (2) are physically separate and co-packaged.

In a further embodiment of the article of manufacture, the solution of (1) comprises the aluminum salt, phosphate, water, and a pH of less than 4; and the solution or dried composition of (2) comprises antigen, buffer (pH 6.0 or greater), sugar and salt.

The present invention further provides a method for making a vaccine comprising combining components (1) and (2) of the above article of manufacture.

The present invention further provides article of manufacture comprising:

(1) a solution comprising an aluminum salt; and

(2) a solution comprising phosphate, an antigen, and base, wherein the phosphate and the base are present in sufficient quantities that when combined with the solution of (1) cause the aluminum and the phosphate to react and precipitate;

wherein each of solutions (1) and (2) are physically separate and co-packaged.

The present invention further provides a method for making a vaccine, comprising combining components (1) and (2) of the above article of manufacture.

The present invention further provides a method for storing components of an adjuvant that is the product of a reaction between an aluminum compound and a phosphate compound under basic conditions comprising storing the aluminum compound and the phosphate compound together under conditions whereby no reaction occurs.

In a further embodiment of the article of manufacture, the conditions whereby no reaction occurs is by

(a) keeping the aluminum compound and the phosphate compound, and, optionally, a base, physically separated; or

(b) storing the aluminum compound and the phosphate compound together at a pH of below about 5.

In a further embodiment of the article of manufacture, (a) comprises storing the aluminum compound in a dried state and the phosphate compound in a dried state in a single vessel or device.

The present invention further provides a method for immunizing a subject comprising combining the components of the article of manufacture of any one of claims 4, 10, 15, 19, 23, and 26 to generate a vaccine formulation and administering the vaccine formulation to a subject. In particular embodiments, a method is used for treating or preventing an infectious disease or cancer, in a subject.

The present invention provides an article of manufacture as disclosed herein wherein the components which are dried are lyophilized, microwave vacuum dried or spray dried. In a further embodiment, the dried component is a lyosphere.

The present invention further provides for the use of the components of the article of manufacture as disclosed herein to generate a vaccine formulation for treating or preventing an infectious disease or cancer in a subject.

The present invention further provides the components of the article of manufacture as disclosed herein for the manufacture of a vaccine formulation for treating or preventing an infectious disease or cancer in a subject.

The present invention also provides an article of manufacture (e.g., a device or vessel) comprising a composition that comprises the components: (1) a dried aluminum adjuvant; and (2) antigen and, optionally, (3) water; wherein either (i) the antigen is dissolved in the water, or (ii) the composition further comprises lyophilized antigen; wherein (1) and (2) and (3) are physically separated and co-packaged. Components (1) and/or (2) and/or (3) optionally further comprise a salt (e.g., NaCl), a buffer, a preservative (e.g. m-cresol or phenol), a surfactant (e.g., polysorbate 20 or polysorbate 80), and/or a sugar (e.g., sucrose or trehalose).

The present invention also provides an article of manufacture comprising a composition comprising the components: (1) lyophilized aluminum salt; (2) lyophilized phosphate; and (3) optionally water and/or antigen (e.g., a protein; a toxin or toxoid; a polysaccharide; a polysaccharide-protein conjugate; a 33-valent pneumococcal polysaccharide conjugated to a protein or proteins; a 21-valent pneumococcal polysaccharide conjugated to a protein (e.g., diphtheria toxin); a 15-valent pneumococcal polysaccharide conjugated to a protein; a 1-valent Type 3 pneumococcal polysaccharide conjugated to a protein; or a 1-valent Type 19A pneumococcal polysaccharide conjugated to a protein) wherein (1), (2) and (3) are physically separated and co-packaged. For example, in an embodiment of the invention, the water does not contain antigen and/or the article further comprises (4) lyophilized antigen which is physically separated from (1), (2) and (3). Any one or more of (1)-(4) may optionally include salt, preservative, buffer, surfactant and/or sugar.

The present invention also provides an article of manufacture comprising a composition comprising: (1) an antigen which is lyophilized or in solution; (2) lyophilized aluminum- and/or calcium-containing reagent; and (3) lyophilized phosphate reagent and, optionally, base; wherein each component (1), (2) and (3) are each physically separated from the other components and co-packaged; and wherein said aluminum- and/or calcium-containing reagent and said phosphate reagent and, optionally, base, react when reconstituted with water to form an adjuvant.

Also provided by the present invention is an article of manufacture comprising a composition comprising: (1) a solution comprising an aluminum salt and a phosphate with a pH of about 5 or less which is substantially clear; and (2) (i) a solution comprising base sufficient to cause said reaction and precipitation when added to (1), and antigen; or (ii) a lyophilized composition which, when reconstituted, comprises base sufficient to cause said reaction and precipitation when added to (1), and antigen; wherein each component (1) and (2) are physically separate and co-packaged.

The present invention also provides an article of manufacture comprising a composition comprising (1) a solution comprising an aluminum salt; and (2) a solution comprising phosphate, antigen and base wherein the phosphate and base are present in sufficient quantities, when combined with (1), to cause the aluminum and phosphate to react and precipitate; wherein each component (1) and (2) are physically separate and co-packaged.

In addition, the present invention also provides a method for storing components of an adjuvant that is the product of a reaction between an aluminum compound and a phosphate compound under basic conditions comprising storing the aluminum compound and the phosphate compound together under conditions whereby no reaction occurs; e.g., wherein the conditions whereby no reaction occurs is: (i) keeping the aluminum compound and the phosphate compound, and, optionally, the base, physically separated; or (ii) storing the aluminum compound and the phosphate compound together at a pH of less than about 4. In an embodiment of the invention, (i) comprises storing the aluminum compound in a lyophilized state and the phosphate compound in a lyophilized state in a single vessel or device.

The present invention also provides methods for making a vaccine formulation including the step of combining the components of any of the articles of manufacture discussed herein. Such a method may further include the step of administering the resulting vaccine formulation to a subject in need thereof. Methods for treating or preventing an infectious disease or cancer, in a subject, by immunizing the subject with the product of such a method is also a product of the method and the product of the method itself is also part of the present invention.

The present invention further provides for use of the components of the article of manufacture disclosed herein to generate a vaccine formulation for treating or preventing an infectious disease or cancer in a subject. The present invention further provides the components of the article of manufacture as disclosed herein for the manufacture of a vaccine formulation for treating or preventing an infectious disease or cancer in a subject.

### BRIEF DESCRIPTION OF THE DRAWINGS

FIG. 1. Class A embodiment. Vials comprising (1) Pre-formed dried aluminum adjuvant and (2) Liquid.

FIG. 2. Static light scattering particle size distribution (Formulation #1, never frozen; Formulation #1, frozen and thawed; Formulation #2, aluminum adjuvant lyobead after reconstitution; and Formulation #3, aluminum adjuvant lyobead after reconstitution).

FIG. 3. Static light scattering particle size distribution (Formulation #2, settled, frozen, thawed; Formulation #2 frozen as a bead, lyophilized, reconstituted).

FIG. 4. Class A embodiment. Vials comprising (1) Pre-formed dried aluminum adjuvant; (2) Formulated Antigen(s); and (3) Liquid.

FIG. 5. Static light scattering particle size distribution (Formulation #1, never frozen; Formulation #3, reconstituted aluminum adjuvant lyobead; Formulation #6A, vaccine from co-reconstituted aluminum adjuvant and antigen; Formulation #6B, vaccine from co-reconstituted aluminum adjuvant and antigen; Formulation #1, frozen and thawed).

FIG. 6. Class B embodiment. Vials containing (1) Dried formulation comprising some of the reactants needed to form aluminum adjuvant; (2) Dried formulation comprising others of the reactants and (3) Liquid.

FIG. 7. Static light scattering particle size distribution (Formulation #1, never frozen; Lyobeads of Formulation #7+8A reconstituted together; Lyobeads of Formulation #7+8B reconstituted together; Formulation #1, frozen and thawed).

FIG. 8. Class B embodiment. Vials comprising (1) Dried formulation comprising some of the reactants needed to form aluminum adjuvant (for example, an aluminum containing compound); (2) Dried formulation comprising other reactants; (3) Liquid; and (4) Formulated Antigen(s).

FIG. 9. Static light scattering particle size distribution (Multivalent vaccine from co-reconstitution of lyobeads of pre-formed aluminum adjuvant and lyobeads of antigen; Multivalent vaccine from co-reconstitution of an aluminum reagent lyobead and a phosphate reagent lyobead which form an aluminum adjuvant, which is then used to reconstitute an antigen lyobead, frozen and thawed aluminum adjuvant (Formulation #1) with no antigens).

FIG. 10. Class B embodiment, and also a Class C embodiment. Vials containing (1) a solution of aluminum- and phosphate-containing compounds with either liquid (2) or lyophilized (3) antigen(s) in a formulation.

FIG. 11. Class B embodiment, and also a Class C embodiment. Vials containing (1) Dried formulation comprising some of the reactants needed to form aluminum adjuvant and (2) Liquid comprising other reactants.

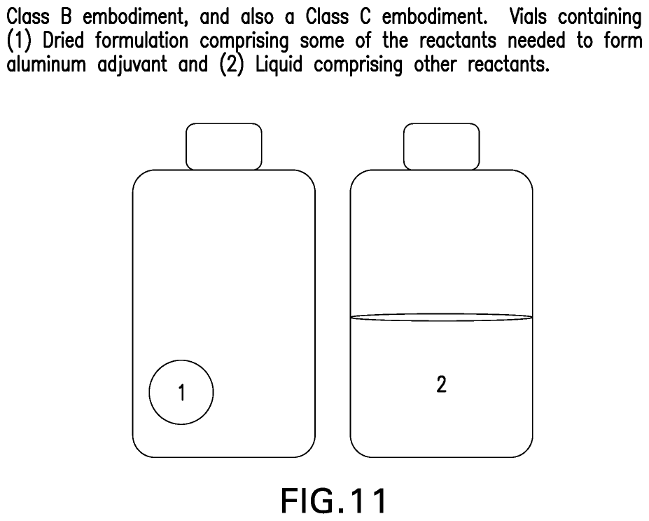

FIG. 12. Class B embodiment. Vials containing (1) Dried formulation comprising some of the reactants needed to form aluminum adjuvant (2) Dried formulation comprising other reactants; (3) Dried formulation comprising Antigen(s); and (4) Liquid.

FIG. 13. Static light scattering particle size distribution (Aluminum adjuvant formed upon reconstitution of lyobeads of Formulation #7 and 8A; Vaccine formed on reconstitution of lyobeads of Formulation #4, 7 and 8A).

FIG. 14. Class C embodiment. Vial containing (1) a solution comprising an aluminum-containing compound and a phosphate-containing compound, but lacking sufficient base to allow completion of the aluminum adjuvant precipitation and (2) a solution comprising a buffer or salt or base that provides the rest of the base needed to allow completion of the aluminum adjuvant precipitation reaction.

FIG. 15. ELISA assay of antigen potency.

FIG. 16. Fluorescence spectra analyses.

FIG. 17. Class C embodiment. Vial containing (1) a solution comprising an aluminum-containing compound and (2) a solution comprising a phosphate-containing compound or buffer or salt or base that provides enough phosphate and/or base to allow completion of the aluminum adjuvant precipitation reaction.

FIG. 18. Class C embodiment. Vial containing (1) a solution of an aluminum-containing compound, and (2) a solution of 4 different antigens (protein bacterial toxins or toxoids) and phosphate pH 8.

*Formulation numbers cited in the Brief Description of the Figures are as described in the Example corresponding to the particular figure.

### DETAILED DESCRIPTION OF THE INVENTION

The vaccine formulations of the present invention (e.g., as provided in an article of manufacture) provide significant advantages over commonly used vaccine formulations and methods of making such formulations. The present invention facilitates the generation of lyophilized vaccine formulations in some embodiments without the need to lyophilize a suspension. Lyophilizing a suspension or lyophilizing a pre-formed complex between antigen and aluminum (Al)- or calcium (Ca)-containing adjuvant presents technical challenges and, typically, it is desirable to avoid such steps. Since Al- or Ca-containing adjuvants form aqueous suspensions, generation of a lyophilized vaccine formulation wherein such an adjuvant is subjected to lyophilization, would be difficult.

The present invention thus provides a method for generating a lyophilized formulation avoiding the need to lyophilize a suspension, i.e., wherein the Al- or Ca-containing component of the adjuvant is lyophilized from a solution and physically separated from the other adjuvant component or reactant—the phosphate-containing reagent or base which is also lyophilized from a solution. Thus, in some embodiments only solutions of the adjuvant components (or reactants) are lyophilized. In other embodiments, freezing-induced agglomeration of a pre-formed aluminum adjuvant is avoided by extremely fast freezing of a small droplet of suspension in a thoroughly resuspended state, as a lyoparticle. In further extensions of either of the above embodiments, the antigen can be physically separated from the adjuvant or adjuvant reactants, avoiding lyophilization of an antigen-adjuvant complex.

The present vaccine formulations are also beneficial because they can be kept physically stable in the event of freezing and thawing. Freezing and thawing of aluminum adjuvants is a common accidental occurrence and can lead to unwanted agglomeration of the adjuvant. When the adjuvant components (or reactants) are lyophilized, freezing of a suspension does not take place and damage due to freezing is avoided. After lyoparticles of aluminum adjuvant suspension are prepared, they are protected from damage associated with subsequent freezing.

“Physically separated” as used herein with reference to lyophilized formulation components being kept physically separated, refers to keeping the components from substantially combining or mixing. For example, lyoparticles of two or three different varieties can be kept in a single vessel and touching one another, yet physically separated since the components are not substantially combining or mixing. In another embodiment of the invention, physically separated components may be kept in separate vessels or devices or separate compartments within such a vessel or device. Physically separated components may be co-packaged into a single article of manufacture, e.g., said separate vessels or devices may be co-packaged.

A lyoparticle is a discrete particle of lyophilized material, for example, taking the form of a bead or sphere or other shape. A lyoparticle may also be referred to as a lyosphere, sphereon or lyobead. In some embodiments, the lyosphere diameter is from about 2 to about 12 mm, preferably from 2 to 8 mm, such as from 2.5 to 6 mm or 2.5 to 5 mm. In some embodiments, the volume of the lyosphere is from about 20 to 550 μL, preferably from 20 to 100 μL, such as from 20 to 50 μL. In embodiments wherein the lyosphere is not substantially spherical, the size of the lyosphere can be described with respect to its aspect ratio, which is the ratio of the longer dimension to the shorter dimension. The aspect ratio of the lyospheres can from 0.5 to 2.5, preferably from 0.75 to 2, such as from 1 to 1.5.

A “subject” is any mammal, e.g., human, non-human primate, mouse, dog, cat, rabbit, cow, or horse.

I. Aluminum Compounds and Adjuvants

Adjuvants of the present invention include aluminum salts or the reaction product of aluminum salts and another compound such as phosphate and/or a base. See, for example, Harlow, E. and D. Lane (1988; Antibodies: A Laboratory Manual Cold Spring Harbor Laboratory) and Nicklas, W. (1992; Aluminum salts. Research in Immunology 143:489-493). An adjuvant which is an aluminum salt can be, for example, hydrated alumina, alumina hydrate, alumina trihydrate (ATH), aluminum hydrate, aluminum trihydrate, Alhydrogel®, Adjuphos®, Superfos, Amphogel®, aluminum (III) hydroxide, aluminum hydroxyphosphate sulfate (also referred to as AAHS and/or MAA), Merck Aluminum Phosphate Adjuvant (MAPA, also referred to as APA), amorphous alumina, trihydrated alumina, or trihydroxyaluminum. MAPA is an aqueous suspension of aluminum hydroxyphosphate. MAPA is manufactured by blending aluminum chloride and sodium phosphate in a 1:1 volumetric ratio to precipitate aluminum hydroxyphosphate. After the blending process, the material is size-reduced with a high-shear mixer to achieve a monodisperse particle size distribution. The product is then diafiltered against physiological saline and steam sterilized.

A dried (e.g., lyophilized) aluminum adjuvant is an aluminum adjuvant prepared as a liquid suspension and then dried. In an embodiment of the invention, the dried aluminum adjuvant is MAPA (Merck aluminum phosphate adjuvant) or MAA (Merck aluminum adjuvant. MAPA is known in the art; see U.S. Pat. No. 9,573,811). MAA is also called “amorphous aluminum hydroxyphosphate sulfate”. See e.g., Caulfield et al. Human Vaccines 3(4):139-145. Another class of aluminum adjuvant that could be used in a pre-formed dried aluminum adjuvant is aluminum hydroxide

A method for the preparation of the MAPA adjuvant includes the steps of: (a) co-mixing a solution of an aluminum salt (Al) with a solution of a phosphate (P) at a defined P/Al molar ratio to precipitate aluminum hydroxyphosphate adjuvant in the presence of a buffer that maintains a constant pH, wherein said co-mixing involves the simultaneous and continuous combining of the solution of an aluminum salt and the solution of a phosphate and the defined P/Al ratio is in the range of 2.0-3.5; and (b) buffer exchanging with a buffer exchange solution to remove at least 80% of any excess phosphate, buffer or other residual salts from the original reactants.

In an embodiment of the invention, the aluminum adjuvant is an aluminum hydroxyphosphate adjuvant, e.g., that has one or more of the following properties:

A zeta potential Point of Zero Charge (PZC) from 4.2 to 6.9, from 4.7 to 6.4, or from 4.7 to 5.4;

An aggregate particle size volume median diameter (d(v,0.5) measured, e.g., by static light scattering) less than 40 microns, and in particular embodiments from 2 to 10 microns or 3 to 6 microns;

A pH within the range of 3.0 to 8.0, or in particular embodiments a pH within the range of 4.5 to 7.2, or a pH within the range of 4.5 to 5.5; or

A P/Al molar ratio measured in the final precipitated solid of 0.6 to 1.2; in certain embodiments the ratio is 0.8 to 1.2 or 0.9 to 1.1.

II. Articles of Manufacture

The present invention provides articles of manufacture for holding and/or administering a vaccine or its components as discussed herein. The components of an article of manufacture typically include the components of a formulation, e.g., a vaccine formulation, which are kept physically separated within the article. Although the components are generally kept physically separated, they are typically co-packaged in a common article. Such an article may be a kit comprising the components. For example, the components may be in one or more vessels and/or one or more devices. Additional packaging material may be used to contain the components of the articles of manufacture, e.g., a box or packing material and/or product literature.

An article of manufacture may include components in one or more vessels such as, for example, vials, e.g., plastic or glass vials, e.g., with a cap. An article of manufacture may, itself, be a vessel or device that contains the components of the formulation. In an embodiment of the invention, the components are in separate compartments within the vessel or device. For example, in an embodiment of the invention, the device is a multi-compartment injection device wherein components are situated in different compartments.

An article of manufacture may include components in one or more devices, such as, for example, injection devices. An injection device may be a device that introduces a substance, such as a vaccine, into the body of a patient via a parenteral route, e.g., intramuscular, subcutaneous or intravenous. For example, an injection device may be a syringe (e.g., pre-filled with a vaccine, such as an auto-injector) which, for example, includes a cylinder or barrel for holding fluid to be injected (e.g., comprising the antibody or fragment or a pharmaceutical composition thereof), a needle for piecing skin and/or blood vessels for injection of the fluid; and a plunger for pushing the fluid out of the cylinder and through the needle bore. In an embodiment of the invention, an injection device that comprises vaccine is an intravenous (IV) injection device. Such a device includes the vaccine in a cannula or trocar/needle which may be attached to a tube which may be attached to a bag or reservoir for holding fluid introduced into the body of the patient through the cannula or trocar/needle. The vaccine may, in an embodiment of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the invention, an injection device is an autoinjector; a jet injector or an external infusion pump. A jet injector uses a high-pressure narrow jet of liquid which penetrate the epidermis to introduce the vaccine to a patient's body. External infusion pumps are medical devices that deliver the vaccine into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

The vaccines may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The vaccines disclosed herein may also be administered by infusion. Examples of well-known implants and modules for administering the vaccines include those disclosed in: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art and those comprising the pharmaceutical compositions of the present invention are within the scope of the present invention.

Methods for administering a vaccine to a subject comprising combining the components of the formulation and injecting the subject, with the resulting vaccine, e.g., with an injection device as discussed herein, form part of the present invention. The step of combining the components and/or injecting the subject may be performed by a clinician or by the subject himself.

III. Formulations

The present invention provides articles of manufacture comprising compositions including components which are co-packaged, e.g., into a vessel or a device. Though co-packaged, the components may be kept physically separated.

As discussed, the present invention includes methods for making a vaccine comprising combining the components of a vaccine formulation of the present invention. When a dried component and a liquid are combined to form a vaccine, they are reconstituted. Reconstitution of a dried component is a skill commonly held in the art. In an embodiment of the invention, reconstitution may include the step of storing or incubating the combined components for a period of time, e.g., at room temperature (e.g., about 20-25° C.) or under refrigeration (e.g., about 4° C.). Incubation or storage may be, for example, for 1 minute, 30 minutes, an hour, 8 hours or 24 hours.

In an embodiment of the invention, no such incubation or storage, after recombination, is performed and the vaccine may be administered to a subject or otherwise used immediately thereafter.

In an embodiment of the invention, reconstitution includes the step of rotating or agitating (e.g., gently) the combined components.

In an embodiment of the invention, reconstitution includes the step of confirming substantially complete dissolution or suspension of all solids with the liquid component, e.g., by visual inspection.

Methods for administering a vaccine to a subject comprising combining the components of the formulation for reconstitution as discussed above and injecting the subject, with the resulting vaccine, e.g., with an injection device as discussed herein, form part of the present invention. The step of reconstituting the components and/or injecting the reconstituted formulation may be performed by a clinician or by the subject himself.

In an embodiment of the invention, as is discussed below, components are kept under conditions whereby there is insufficient base to cause reaction of an aluminum compound so as to form an aluminum adjuvant which may precipitate (e.g., under acidic pH conditions such as about 4 or less). Sufficient base and the reaction to form the adjuvant occur upon combination of the components. Such lack of a precipitate is apparent when the solution remains substantially clear. A “substantially clear” solution would have an optical density of about 0, e.g., between the ultraviolet and visible wavelengths of light (e.g., 260 nM, 280 nm or 600 nM); and/or upon visual inspection would be substantially free of particulate matter.

In general, the present invention includes three classifications of adjuvanted vaccine:

(A) Involving lyobeads made from a pre-formed aluminum adjuvant;

(B) Involving a dried form of a compound (for example, a lyobead) that reacts upon reconstitution to form an aluminum adjuvant; and

(C) Involving a liquid formulation of a compound that reacts when other necessary reactants are provided to form an aluminum adjuvant.

Some examples may be members of more than one class. Each embodiment classification (A, B and C) is discussed further herein.

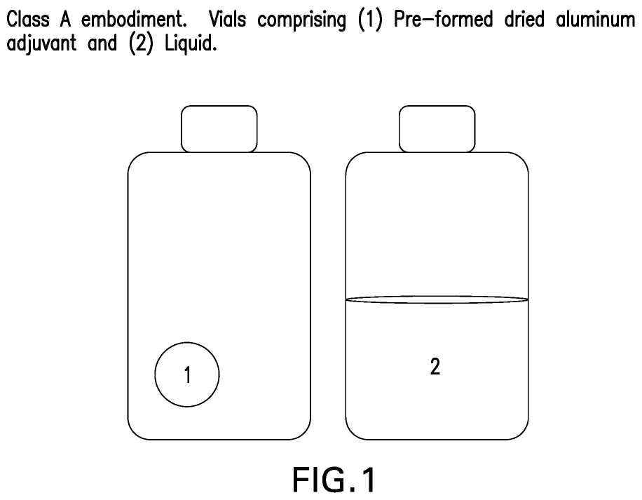

An example of an (A) embodiment of the invention relates to aluminum adjuvant lyobeads prepared from pre-made aluminum adjuvant. See for example, FIG. 1. The present invention includes articles of manufacture comprising such an aluminum adjuvant and, optionally, liquid. Such an article of manufacture includes the aluminum adjuvant (for example, dried aluminum adjuvant, e.g., one or more lyobeads) which are kept physically separated from, but co-packaged with, the optional liquid which may be used for reconstituting the adjuvant. The liquid may optionally be part of the article of manufacture or not. Such liquid may comprise: water; and optionally salt(s), buffer(s), surfactant(s) and/or excipients; optionally antigen(s) (e.g., polysaccharide-protein conjugated antigen); optionally additional adjuvant(s); and/or optionally preservative(s)

In an embodiment of the invention, the liquid comprises water and the antigen and, optionally, one or more other ingredients.

The present invention includes methods for forming a vaccine comprising combining the components of the article of manufacture as well as the product of such a method. Methods for administering a vaccine to a subject (e.g., a human) comprising combining the components and injecting the vaccine into the subject (e.g., with an injection device as discussed herein) also form part of the present invention.

For example, such an embodiment comprises dried (e.g., one or more lyobeads) MAPA with the pre-lyophilized composition, for example, as set forth below (concentrations before drying, e.g., lyophilization):

aluminum adjuvant; and salt or aluminum adjuvant, sugar, salt and base (e.g., an amino acid such as histidine or histidine base); or 1250 mcg Al/mL aluminum adjuvant; 150 mM sodium chloride, or 625-1250 mcg Al/mL of MAPA; 16-23% (w/v) trehalose; 35-70 mM NaCl; 0.5-0.8 mM Histidine base; or for example comprising: 1250 mcg Al/mL aluminum adjuvant; 16% (w/v) trehalose; 70 mM sodium chloride; and, 0.5 mM histidine base; or 625 mcg Al/mL aluminum adjuvant; 23% (w/v) trehalose; 35 mM sodium chloride; and, 0.8 mM histidine base.

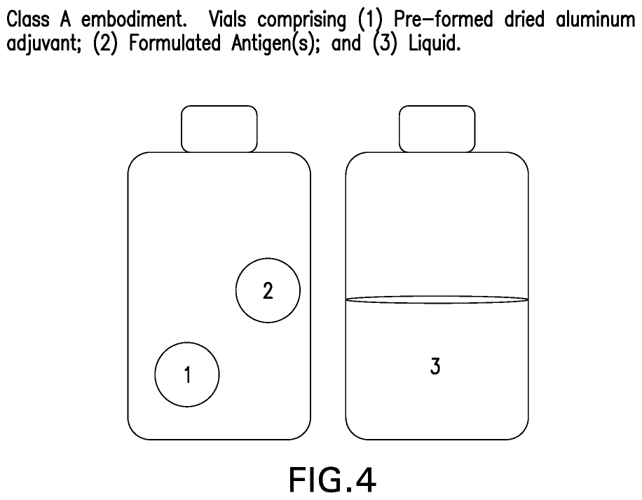

Another example of a class A embodiment of the invention relates to preparation of vaccines by co-reconstitution of dried aluminum adjuvant, e.g., lyobeads (prepared from pre-made aluminum adjuvant), and dried antigen, e.g., one or more lyobeads, without significant freezing or thawing induced agglomeration. See e.g., FIG. 4.

In such an embodiment of the invention, the following three components are kept physically separated: pre-formed dried aluminum adjuvant (example: lyobeads); and formulated antigen(s) and liquid which may comprise: water; optionally salt(s), buffer(s), surfactant(s) and/or excipients (e.g., phosphate buffer, histidine buffer, dextrose or NaCl as tonicity modifier); optionally antigen(s); optionally additional adjuvant(s) (e.g., one or more immune stimulatory molecules, for example, as set forth herein); and optionally one or more preservatives.

As defined herein, an “adjuvant” is a substance that serves to enhance the immunogenicity of an immunogenic composition of the invention. An immune adjuvant may enhance an immune response to an antigen that is weakly immunogenic when administered alone, e.g., inducing no or weak antibody titers or cell-mediated immune response, increase antibody titers to the antigen, and/or lower the dose of the antigen effective to achieve an immune response in the individual. Thus, adjuvants are often given to boost the immune response and are well known to the skilled artisan. Suitable adjuvants to enhance effectiveness of the composition include, but are not limited to:

(1) aluminum salts (alum), such as aluminum hydroxide, aluminum phosphate, aluminum sulfate, etc.;

(2) oil-in-water emulsion formulations (with or without other specific immunostimulating agents such as muramyl peptides (defined below) or bacterial cell wall components), such as, for example, (a) MF59 (International Patent Application Publication No. WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, Mass.), (b) SAF, containing 10% Squalene, 0.4% Tween 80, 5% pluronic-blocked polymer L121, and thr-MDP either microfluidized into a submicron emulsion or vortexed to generate a larger particle size emulsion, (c) Ribi™ adjuvant system (RAS), (Corixa, Hamilton, Mont.) containing 2% Squalene, 0.2% Tween 80, and one or more bacterial cell wall components from the group consisting of 3-O-deaylated monophosphorylipid A (MPL™) described in U.S. Pat. No. 4,912,094, trehalose dimycolate (TDM), and cell wall skeleton (CWS), preferably MPL+CWS (Detox™); and (d) a Montanide ISA;

(3) saponin adjuvants, such as Quil A or STIMULON™ QS-21 (Antigenics, Framingham, Mass.) (see, e.g., U.S. Pat. No. 5,057,540) may be used or particles generated therefrom such as ISCOM (immunostimulating complexes formed by the combination of cholesterol, saponin, phospholipid, and amphipathic proteins) and Iscomatrix® (having essentially the same structure as an ISCOM but without the protein);

(4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-tetradecanoyloxytetradecanoylamino]ethyl 2-Deoxy-4-O-phosphono-3-O-[(R)-3-tetradecanoyloxytetradecanoyl]-2-[(R)-3-tetradecanoyloxytetradecanoylamino]-b-D-glucopyranoside, which is also known as 529 (formerly known as RC529), which is formulated as an aqueous form or as a stable emulsion;

(5) synthetic polynucleotides such as oligonucleotides containing CpG motif(s) (U.S. Pat. No. 6,207,646);

(6) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc.; and/or

(7) complement, such as a trimer of complement component C3d.

In another embodiment, the adjuvant is a mixture of 2, 3, or more of the above adjuvants, e.g., SBAS2 (an oil-in-water emulsion also containing 3-deacylated monophosphoryl lipid A and QS21).

Muramyl peptides include, but are not limited to, N-acetyl-muramyl-L-threonyl-D-isoglutamine (thr-MDP), N-acetyl-normuramyl-L-alanine-2-(1′-2′ dipalmitoyl-sn-glycero-3-hydroxyphosphoryloxy)-ethylamine (MTP-PE), etc.

In certain embodiments, as is discussed above, the adjuvant is an aluminum salt. The aluminum salt adjuvant may be an alum-precipitated vaccine or an alum-adsorbed vaccine. In one embodiment, the vaccine is pre-absorbed on the aluminum adjuvant. Aluminum-salt adjuvants are well known in the art and are described, for example, in Harlow, E. and D. Lane (1988; Antibodies: A Laboratory Manual Cold Spring Harbor Laboratory) and Nicklas, W. (1992; Aluminum salts. Research in Immunology 143:489-493). The aluminum salt includes, but is not limited to, hydrated alumina, alumina hydrate, alumina trihydrate (ATH), aluminum hydrate, aluminum trihydrate, Adjuphos®, Alhydrogel®, Superfos, Amphogel®, aluminum (III) hydroxide, aluminum hydroxyphosphate sulfate (sometimes referred to as AAHS or

MAA) Merck Aluminum Phosphate Adjuvant (MAPA), amorphous alumina, trihydrated alumina, or trihydroxyaluminum.

An article of manufacture comprising the adjuvant, antigen and liquid as discussed above forms part of the present invention. The present invention includes methods for making a vaccine comprising combining the adjuvant, antigen and liquid as well as the product of such a method. Methods for administering a vaccine to a subject (e.g., a human) comprising combining the components and injecting the vaccine into the subject (e.g., with an injection device as discussed herein) also form part of the present invention.

In an embodiment of the invention, such an aluminum adjuvant of the present invention comprises: dried (e.g., one or more lyobeads) MAPA, e.g., comprising, pre-lyophilization: (i) 1250 mcg Al/mL aluminum adjuvant; 16% (w/v) trehalose; 70 mM sodium chloride; and, 0.5 mM histidine base; or, (ii) 625 mcg Al/mL aluminum adjuvant; 23% (w/v) trehalose; 35 mM sodium chloride; and, 0.8 mM histidine base.

In an embodiment of the invention, such an antigen comprises: PCV antigen, e.g., formulations including: 21-valent, 33-valent, 15-valent, 1-valent Type 3 and/or 1-valent Type 19A pneumococcal polysaccharide protein conjugate antigen, e.g., wherein the antigen component comprises the formulation, before drying: 320 mcg PnPs/mL CRM-PnPs (pneumococcal polysaccharide) CRM-PnPs conjugate antigen(s); 100 mM histidine; 20% (w/v) trehalose; 50 mM sodium chloride; and 0.1% polysorbate 20; at pH 6-7.5.

In an embodiment of the invention, the vaccine comprises one antigen (“mono-valent”) or more than one antigen (“multi-valent”, or “combination”) which may be prepared by co-packaging dried (e.g., one or more lyobeads) aluminum adjuvant with dried (e.g., one or more lyobeads) formulated antigen or antigens. In an embodiment of the invention, the aluminum adjuvant component comprises, before drying, e.g., lyophilization (i) 1250 mcg Al/mL aluminum adjuvant; 150 mM sodium chloride; and 23% (w/v) trehalose, sucrose and/or combination thereof; or, (ii) 625 mcg Al/mL aluminum adjuvant; 23% (w/v) trehalose; 35 mM sodium chloride; and, 0.8 mM histidine base.

In an embodiment of the invention, the antigen comprises, before drying, e.g., lyophilization, 20% (w/v) trehalose; 50 mM histidine (base); 50 mM histidine hydrochloride; 320 mcg PnPs/mL CRM-PnPs (Pneumococcal polysaccharide) conjugate antigen, type 19A; 50 mM sodium chloride; and 0.1% polysorbate 20, at pH 6.

In an embodiment of the invention, the antigen is a multi-antigen composition comprising, before drying, e.g., lyophilization, 20% (w/v) trehalose; 100 mM histidine (base); 320 mcg PnPs/mL CRM-PnPs (Pneumococcal polysaccharide) conjugate antigen, a blend of 15 conjugates with different types of polysaccharides; 50 mM sodium chloride; and 0.1% polysorbate 20; at pH 7.5.

In an embodiment of the invention, the formulations, after reconstitution of dried aluminum adjuvant (e.g., one or more lyobeads) and dried antigen (e.g., one or more lyobeads) with liquid, comprises 13.2% (w/v) trehalose; 250 mcg Al/mL aluminum adjuvant; 64 mcg PnPs/mL CRM-PnPs conjugate antigen, all type 19A (A) or a blend comprising 15 PnPs types (B); 24 mM sodium chloride; 20.3 mM histidine (base); and 0.02% polysorbate 20.

An example of a class B embodiment of the invention relates to separate lyobeads of aluminum containing solution and phosphate/base, which are physically separate (and, optionally, co-packaged into a single vessel) and liquid which is also physically separate from the lyobeads, e.g., in a second, separate vessel. The aluminum and phosphate/base may react to form aluminum adjuvant in a vial or in a reconstitution chamber when combined with liquid.

In such an embodiment of the invention, some of the reactants (e.g., the following three reactants) are kept physically separate from others of the reactants, and some or all of the reactants are dried. For example, a dried formulation (e.g., one or more lyobeads) comprising some of the reactants needed to form the aluminum adjuvant (for example, an aluminum containing compound); and a dried formulation (e.g., one or more lyobeads) comprising the other reactants needed to form aluminum adjuvant (for example, a phosphate containing compound and/or a base) and liquid, e.g., comprising: water; optionally salt(s), buffer(s), surfactant(s) and/or excipients; optionally antigen(s); optionally additional adjuvant(s); and/or optionally preservative(s).

An article of manufacture comprising the physically separated aluminum containing component, the phosphate/base containing component and the liquid as discussed above forms part of the present invention. Methods for making a vaccine comprising combining the aluminum, phosphate/base and liquid forms part of the present invention along with the product of such a process. Methods for administering a vaccine to a subject (e.g., a human) comprising combining the components and injecting the vaccine into the subject (e.g., with an injection device as discussed herein) also form part of the present invention.

In an embodiment of the invention, the aluminum containing component comprises, before drying, e.g., lyophilization: 92.66 mM aluminum chloride and 25% (w/v) trehalose.

In an embodiment of the invention, the phosphate/base containing component comprises, before drying, e.g., lyophilization: 92.66 mM sodium phosphate tribasic and 25% (w/v) trehalose; or 92.47 mM sodium phosphate tribasic and 24.95% (w/v) trehalose; or 92.29 mM sodium phosphate tribasic and 24.9% (w/v) trehalose.

Upon reconstitution, aluminum adjuvant can be formed by the reaction of the aluminum compound with a phosphate compound or base, when enough base is provided for a precipitation reaction, resulting in formation of the aluminum adjuvant, to occur.

In an embodiment of the invention, the aluminum adjuvant formed upon reconstitution with the phosphate/base comprises: (i) 1250 mcg Al/mL aluminum adjuvant; 150 mM sodium chloride; and 16% (w/v) trehalose, sucrose and/or combination thereof; or (ii) 1250 mcg Al/mL aluminum adjuvant; 16% (w/v) trehalose; 70 mM sodium chloride; and 0.5 mM histidine base.

In an embodiment of the invention, the aluminum containing component comprises, before drying, e.g., lyophilization: 92.66 mM aluminum chloride; and, 25% (w/v) trehalose.

In an embodiment of the invention, the phosphate/base containing component comprises, before drying, e.g., lyophilization: 92.66 mM sodium phosphate tribasic; and, 25% (w/v) trehalose.

In an embodiment of the invention, the phosphate/base containing component comprises, before drying, e.g., lyophilization: 92.47 mM sodium phosphate tribasic; 24.95% (w/v) trehalose; and, 10 mM sodium hydroxide.

In an embodiment of the invention, the phosphate/base containing component comprises, before drying, e.g., lyophilization: 92.29 mM sodium phosphate tribasic; 24.9% (w/v) trehalose; and 20 mM sodium hydroxide.

Another example of a class B embodiment of the invention relates to preparation of vaccines (aluminum adjuvant and antigen) by reconstitution of physically separate lyobeads of aluminum containing solution and phosphate/base containing solution (e.g., which are co-packaged) which react to form aluminum adjuvant in a vial (or in a reconstitution chamber) when combined with liquid. Such a newly formed aluminum adjuvant suspension may be used to reconstitute antigen lyobeads to form a complete vaccine.

In such an embodiment of the invention, the following four components are kept physically separate: dried formulation (e.g., one or more lyobeads) comprising some of the reactants needed to form aluminum adjuvant (for example, an aluminum containing compound); and dried formulation (e.g., one or more lyobeads) comprising others of the reactants needed to form aluminum adjuvant (for example, a phosphate containing compound and/or a base) which may be co-packaged; and liquid comprising: (i) water; optionally salt(s), buffer(s), surfactant(s) and/or excipients; optionally antigen(s); optionally additional adjuvant(s); and/or optionally preservative(s); and (ii) lyophilized (e.g., lyobead) antigen.

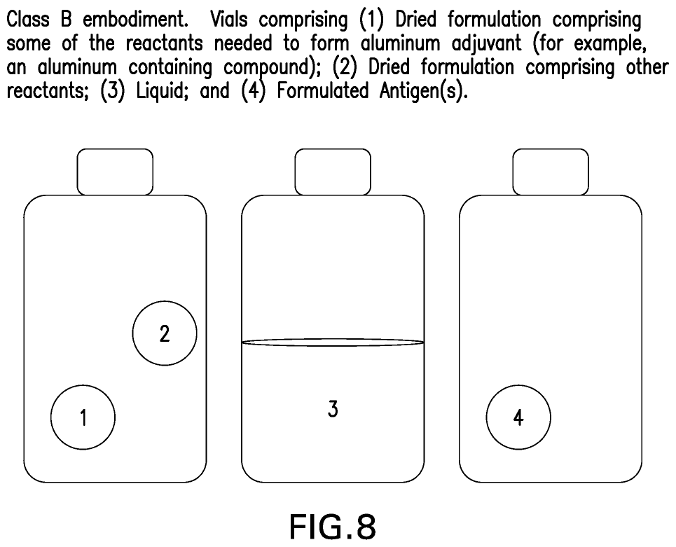

In a further embodiment of an article of manufacture of the present invention, the dried components, comprising the reactants, are in a common vessel; the dried antigen is in a second vessel; and the liquid is in a third vessel. See e.g., FIG. 8.

The present invention includes an article of manufacture comprising the two dried components, which are physically separated but may be co-packaged, and the liquid (physically separated from the other components) and the antigen (physically separated from the other components). Methods for making a vaccine comprising combining said components also forms part of the present invention as well as the product of such a method. Methods for administering a vaccine to a subject (e.g., a human) comprising combining the components and injecting the vaccine into the subject (e.g., with an injection device as discussed herein) also form part of the present invention.

In an embodiment of the invention, the aluminum containing component comprises, before drying e.g., before lyophilization: 92.66 mM aluminum chloride and 25% trehalose (w/v).

In an embodiment of the invention, the phosphate/base containing component comprises, before drying, e.g., before lyophilization: 92.66 mM sodium phosphate tribasic and 25% (w/v) trehalose.

In an embodiment of the invention, the antigen containing component comprises a 15-valent antigen formulation that comprises, before drying e.g., before lyophilization: 320 mcg PnPs/mL CRM-PnPs (pneumococcal polysaccharide) conjugate antigens; 50 mM histidine base; 50 mM histidine hydrochloride; 20% (w/v) trehalose; 50 mM sodium chloride; and 0.1% polysorbate 20; at pH 6.

In an embodiment of the invention, the aluminum-containing component comprises before drying e.g., before lyophilization 92.66 mM aluminum chloride; and, 25% (w/v) trehalose.

In an embodiment of the invention, the aluminum adjuvant component formed by combination of the aluminum and phosphate/base containing component comprises upon reconstitution: 625 mcg Al/mL aluminum adjuvant; 23% (w/v) trehalose; 35 mM sodium chloride; and 0.8 mM histidine base.

In an embodiment of the invention, the phosphate/base containing component comprises, before drying, e.g., before lyophilization: 92.66 mM sodium phosphate tribasic; and, 25% (w/v) trehalose.

In an embodiment of the invention, the antigen containing component comprises, before drying e.g., before lyophilization: 20% (w/v) trehalose; 50 mM histidine (base); 50 mM histidine hydrochloride; 320 mcg PnPs/mL CRM-PnPs (pneumococcal polysaccharide) CRM-PnPs conjugate antigens; 50 mM sodium chloride; and 0.1% polysorbate 20; at pH 6

Another embodiment of the invention relates to preparation of monovalent and multivalent HPV vaccines by combining a liquid solution of aluminum- and phosphate-containing compounds (that has not yet reacted to form an aluminum adjuvant suspension) with formulated antigen in either the liquid (class C embodiment) or lyophilized (both class B and class C embodiment) state, resulting in reaction to form aluminum adjuvant in the presence of one or more antigens.

In such an embodiment of the invention, the following components are kept physically separate:

(1) a solution of aluminum- and phosphate-containing compounds lacking sufficient base (e.g., at a pH of about 5 or less, e.g., about 1, 1.5, 2, 2.5, 3, 3.5, 4, 4.5, or 5) to complete the reaction to form aluminum adjuvant suspension and either

(2) a liquid that can serve as a source of base which may comprise antigen or

(3) dried, e.g., lyophilized, antigen(s); wherein the formulated antigen comprises a buffer, salt and/or base that allows completion of the aluminum adjuvant precipitation reaction in the presence of antigen(s).

The present invention includes articles of manufacture comprising either: (i) physically separate (1) and (2) as set forth above; or (ii) physically separate (1) and (3) as set forth above.

Methods for forming a vaccine comprising combining (1) and (2); or (1) and (3) are part of the present invention as well as the product of such a method. Methods for administering a vaccine to a subject (e.g., a human) comprising combining the components and injecting the vaccine into the subject (e.g., with an injection device as discussed herein) also form part of the present invention.

In an embodiment of the invention,

(1) comprises a solution obtained by mixing equal volumes of 37.08 mM aluminum chloride in water with 33.38 mM sodium phosphate pH 7.0 in water, resulting in 18.54 mM aluminum chloride, 16.69 mM sodium phosphate, at acidic pH (e.g., about 4 or less);

(2) comprises (50, 80, or 75) mM sodium phosphate dibasic, (50, 20, or 25) mM sodium phosphate monobasic, 160 mcg/mL of HPV VLP type 16, 5 mM histidine, 125 mM sodium chloride, 0.005% polysorbate 80, in water; and/or

(3) comprises (20 or 42.5) mM histidine base, 4.5% sucrose, HPV VLP types 6 (40 mcg/mL), 11 (80 mcg/mL), 16 (80 mcg/mL), and 18 (40 mcg/mL), 125 mM sodium chloride, 0.005% polysorbate 80, lyophilized.

A solution of aluminum- and phosphate-containing compounds (lacking sufficient base (e.g., at a pH of about 5 or less, e.g., about 1, 1.5, 2, 2.5, 3, 3.5, 4, 4.5, or 5) to complete the reaction to form aluminum adjuvant suspension) can be mixed with formulated HPV virus-like particles of a single VLP type or multiple VLP types. Buffers present with the formulated antigens may provide the base that was lacking to allow reaction to form aluminum adjuvant in the presence of the antigens. These reactions may be performed at small volumes (for example 1 mL added to 1 mL) as could be the case in vaccine vials or delivery devices.

In an embodiment of the invention, the aluminum and phosphate containing component (1) lacking sufficient base (e.g., at a pH of about 5 or less, e.g., about 1, 1.5, 2, 2.5, 3, 3.5, 4, 4.5, or 5) to form the aluminum adjuvant comprises: (i) 18.54 mM aluminum chloride hexahydrate; 16.69 mM sodium phosphate; and in water, at acidic pH; or (ii) 9.27 mM aluminum chloride hexahydrate; 8.34 mM sodium phosphate; and in water, at acidic pH.

In an embodiment of the invention, the antigen (3), before drying, e.g., lyophilization, comprises:

(A)(i) 50 mM sodium phosphate dibasic; 50 mM sodium phosphate monobasic; 160 mcg/mL of HPV VLP type 16; 5 mM histidine; 125 mM sodium chloride; and, 0.005% polysorbate 80; in water; or (ii) 80 mM sodium phosphate dibasic; 20 mM sodium phosphate monobasic; 160 mcg/mL of HPV VLP type 16; 5 mM histidine; 125 mM sodium chloride; and 0.005% polysorbate 80; in water; or

(B)(i) 75 mM sodium phosphate dibasic; 25 mM sodium phosphate monobasic; 160 mcg/mL of HPV VLP type 16; 5 mM histidine; 125 mM sodium chloride; and, 0.005% polysorbate 80; in water; or (ii) 20 mM histidine base; 4.5% sucrose; 40 mcg/mL of HPV VLP type 6; 80 mcg/mL of HPV VLP type 11; 80 mcg/mL of HPV VLP type 16; 40 mcg/mL of HPV VLP type 18; 125 mM sodium chloride; 0.005% polysorbate 80; in water, or

(C)(i) 42.5 mM histidine base; 4.5% sucrose; 40 mcg/mL of HPV VLP type 6; 80 mcg/mL of HPV VLP type 11; 80 mcg/mL of HPV VLP type 16; 40 mcg/mL of HPV VLP type 18; 125 mM sodium chloride; and 0.005% polysorbate 80; in water.

Another embodiment that fits in both class B and class C relates to preparation of aluminum adjuvant by combination of a liquid solution comprising aluminum-containing compounds and phosphate-containing compounds (said solution does not, by itself, react to form an aluminum adjuvant suspension with precipitated solids), with a dried (e.g., lyophilized or spray-dried) formulation which provides the base needed to complete the aluminum adjuvant formation reaction.

In such an embodiment of the invention, the following two components are kept physically separate: dried (e.g., lyophilized or spray-dried) formulation comprising some of the reactants needed to form aluminum adjuvant (for example, a formulated antigen with phosphate and/or buffer and/or base, optionally buffer and/or excipients, optionally additional adjuvants, optionally preservative(s)) and liquid, comprising: water; other reactants needed to form aluminum adjuvant (for example, an aluminum-containing compound and a phosphate-containing compound, lacking sufficient base (e.g., at a Ph of about 5 or less, e.g., about 1, 1.5, 2, 2.5, 3, 3.5, 4, 4.5, or 5) for completion of the aluminum adjuvant precipitation reaction); optionally buffer(s), salt(s), surfactant(s) and/or excipient(s); optionally additional adjuvant(s); and/or optionally preservative(s).

The present invention provides an article of manufacture comprising the physically separated dried (e.g., lyophilized or spray-dried) formulation and liquid as well as a method for making a vaccine comprising combining said components as well as the product of such a method. Methods for administering a vaccine to a subject (e.g., a human) comprising combining the components and injecting the vaccine into the subject (e.g., with an injection device as discussed herein) also form part of the present invention.

For example, in an embodiment of the invention, the dried (e.g., spray-dried or lyophilized) formulations comprise, before drying, 1% sucrose, 9% mannitol, 50 mM phosphate, pH 7.4 or pH 7.8; or, before drying (e.g., spray-dried or lyophilized), 6% sucrose, 100 mM histidine, pH 6.0 or 7.0 or 7.4

In an embodiment of the invention, the liquid component comprises 10.49 mM aluminum chloride, 9.44 mM sodium phosphate, in water at acidic pH.

In an embodiment of the invention, the liquid comprises: 10.49 mM aluminum chloride; 9.44 mM sodium phosphate; in water, at acidic pH

In an embodiment of the invention, the dried component comprises, before drying (e.g., lyophilization or spray drying): (i) 6% sucrose; 100 mM histidine; pH 7.6, lyophilized; or (ii) 6% sucrose; 100 mM histidine; pH 7.0, lyophilized; or (iii) 6% sucrose; 100 mM histidine; pH 6.0, lyophilized; or (iv) 1% sucrose; 9% mannitol; 50 mM phosphate; pH 7.4, spray dried; or (v) 1% sucrose; 9% mannitol; 50 mM phosphate; pH 7.8, spray dried.

Another class B embodiment relates to preparation of a vaccine by co-packaging of lyobeads of an aluminum-containing solution, a phosphate/base-containing solution, and formulated antigen and liquid to allow reaction and formation of aluminum adjuvant in the presence of antigen.

In such an embodiment of the invention, the following components are kept physically separated: dried (e.g., lyophilized) formulation comprising some of the reactants needed to form aluminum adjuvant (for example, an aluminum containing compound); and dried (e.g., lyophilized) formulation comprising others of the reactants needed to form aluminum adjuvant (for example, a phosphate containing compound and/or a base); and dried (e.g., lyophilized) formulation comprising antigen(s), wherein any of which optionally comprises a preservative; and liquid comprising: water; optionally buffer(s), salt(s), surfactant(s) and/or excipients; optionally antigen(s); optionally additional adjuvant(s); and optionally preservative(s).

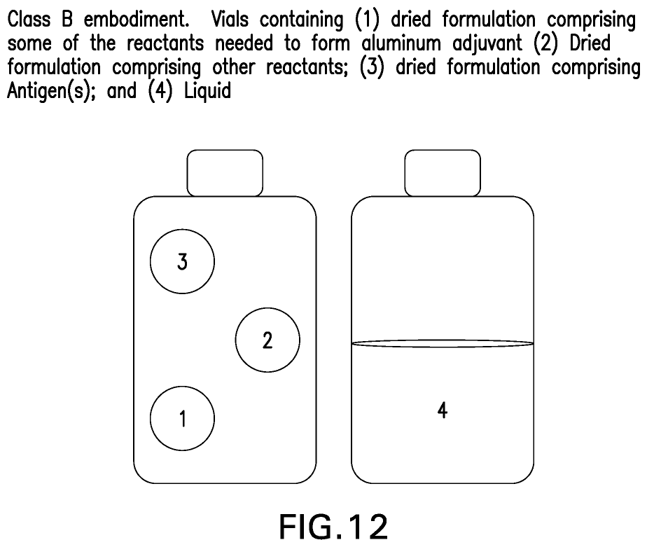

In a preferred embodiment of an article of manufacture of the present invention, all dried components are packaged in a common vessel whereas the liquid component is in a separate vessel. See e.g., FIG. 12.

The present invention provides an article of manufacture comprising said dried (e.g., lyophilized) formulations and liquid all kept physically separated from one another as well as a method for making a vaccine comprising combining said components and, also, the product of such a method. Methods for administering a vaccine to a subject (e.g., a human) comprising combining the components and injecting the vaccine into the subject (e.g., with an injection device as discussed herein) also form part of the present invention.

In an embodiment of the invention, the aluminum-containing component comprises, before drying, e.g., lyophilization, 92.66 mM aluminum chloride and 25% (w/v) trehalose.

In an embodiment of the invention, the phosphate-containing component comprises, before drying, e.g., lyophilization, 92.66 mM sodium phosphate tribasic and 25% (w/v) trehalose.

In an embodiment of the invention, the antigen-containing component comprises, before drying, e.g., lyophilization, 320 mcg PnPs/mL CRM-PnPs (pneumococcal polysaccharide) conjugate antigen (polysaccharide type 19A); 100 mM histidine; 20% (w/v) trehalose; 50 mM sodium chloride; and 0.1% polysorbate 20; at pH 7.5.

In an embodiment of the invention, the antigen-containing component comprises, before drying, e.g., lyophilization, 20% (w/v) trehalose; 100 mM histidine (base); 320 mcg PnPs/mL CRM-PnPS (pneumococcal polysaccharide) conjugate antigen (polysaccharide type 19A); 50 mM sodium chloride; and 0.1% polysorbate 20.

In an embodiment of the invention, the aluminum-containing component comprises, before drying, e.g., lyophilization, 92.66 mM aluminum chloride and 25% (w/v) trehalose.

In an embodiment of the invention, the phosphate-containing component comprises, before drying, e.g., lyophilization, 92.66 mM sodium phosphate tribasic and 25% (w/v) trehalose.

Class C embodiments of the present invention involve a liquid formulation of a compound that reacts when combined with other necessary reactants (liquid or dried) to form an aluminum adjuvant. For example, two liquid solutions can be added to a vessel that results in the formation of aluminum adjuvant (optionally in the presence of antigen(s), salt(s), buffer(s), excipient(s), detergent(s), surfactant(s), preservative(s), stabilizer(s), dye(s), indicator(s)).

For example, in an embodiment of the invention, the following two components are kept physically separated: an aluminum/phosphate-containing component comprising: a solution comprising an aluminum-containing compound and a phosphate-containing compound, but lacking sufficient base (e.g., at a pH of about 5 or less, e.g., about 1, 1.5, 2, 2.5, 3, 3.5, 4, 4.5, or 5) to allow completion of the aluminum adjuvant precipitation reaction; and a component comprising (e.g., an equal volume of) a solution comprising a buffer or salt or base that provides the rest of the base needed to allow completion of the aluminum adjuvant precipitation reaction, and optionally comprising antigen(s) and/or other components.

The present invention provides an article of manufacture comprising the aluminum/phosphate-containing component and the buffer/salt/base-providing component kept physically separate. Methods for making a vaccine comprising combining said components also forms part of the present invention along with the product of such a method. Methods for administering a vaccine to a subject (e.g., a human) comprising combining the components and injecting the vaccine into the subject (e.g., with an injection device as discussed herein) also form part of the present invention.

In an embodiment of the invention, the aluminum/phosphate-containing component comprises: 18.54 mM aluminum chloride (from aluminum chloride hexahydrate) and 16.69 mM sodium phosphate, in water at acidic pH (e.g., prepared from mixture of equal volumes of 37.08 mM aluminum chloride and 33.38 mM sodium phosphate pH 7).

In an embodiment of the invention, the buffer/salt/base-providing component comprises 128 mcg PnPs/mL CRM-PnPs (pneumococcal polysaccharide) conjugate antigen(s), e.g., a blend of conjugates with 15 different polysaccharide types; 80 mM histidine, pH 7.0; 6% sucrose; 150 mM sodium chloride; water

In an embodiment of the invention, the aluminum/phosphate containing component comprises 18.54 mM aluminum chloride hexahydrate; 16.69 mM sodium phosphate; in water; at acidic pH.

In an embodiment of the invention, the product after combination of the aluminum/phosphate containing component and the base-providing component comprises 64 mcg PnPs/mL CRM-PnPS (pneumococcal polysaccharide) conjugate antigens (e.g., a blend of conjugates with 15 different polysaccharide types); 40 mM histidine; 3% sucrose; 100 mM sodium chloride; 250 mcg Al/mL aluminum adjuvant; in water; pH 5.9.

Another class C embodiment of the present invention relates to preparation of aluminum adjuvant by rapid, sequential addition of an aluminum-containing solution and a phosphate/base-containing solution, e.g., on an automated vial filling line. In an embodiment of the invention, two liquid solutions are filled into a vial, e.g., using a filling line, that results in the formation of aluminum adjuvant (optionally in the presence of antigen(s), salt(s), buffer(s), excipient(s), detergent(s), surfactant(s), preservative(s), stabilizer(s), dye(s), indicator(s)), followed immediately by stoppering of the vial.

In such an embodiment of the invention, the following components are kept physically separate: an aluminum-containing component including: a solution comprising an aluminum-containing compound; and a phosphate/base-containing component including, e.g., an equal volume of, a solution comprising a phosphate-containing compound or buffer or salt or base that provides enough phosphate and/or base to allow completion of the aluminum adjuvant precipitation reaction, and optionally comprising antigen(s) and/or other components. Solutions may be added in reverse order, and volumes of addition could be designed intentionally to not be equal if desired.

The present invention provides an article of manufacture comprising the aluminum-containing component and the phosphate/base-containing component which are kept physically separate. In an embodiment of the invention, the article of manufacture is an apparatus for filling vials, e.g., in a laboratory or factory setting which comprises one or more reservoirs that each contain the physically separated components. The present invention also provides a method for making a vaccine comprising combining said components as well as the product of such a method. Methods for administering a vaccine to a subject (e.g., a human) comprising combining the components and injecting the vaccine into the subject (e.g., with an injection device as discussed herein) also form part of the present invention.

In an embodiment of the invention, the aluminum-containing component comprises 18.53 mM aluminum chloride in water.

In an embodiment of the invention, the phosphate/base-containing component comprises 100 mM sodium phosphate, in water, pH 8.1; or alternatively, 100 mM sodium phosphate, 9% (w/v) sucrose, in water, pH 8.1; or 100 mM Tris (i.e., tris(hydroxymethyl)aminomethane), 18.53 mM sodium phosphate, in water, pH 8.1.

In an embodiment of the invention, the aluminum-containing component comprises 18.53 mM aluminum chloride in water.

In an embodiment of the invention, the phosphate/base-containing component comprises (i) 100 mM sodium phosphate, pH 8.1, in water, or (ii) 100 mM sodium phosphate; 9% (w/v) sucrose; pH 8.1, in water, or (iii) 100 mM Tris (also known as tris(hydroxymethyl)aminomethane); 18.53 mM sodium phosphate; pH 8.1, in water.

A further class C embodiment of the invention relates to preparation of a multi-antigen aluminum adjuvanted vaccine, e.g., prepared with a final volume of about 12 mL which could correspond to the volume of a multi-dose container of vaccine, by mixture of a solution of aluminum-containing compound with a second solution that comprised phosphate/base and antigens, resulting in reaction and formation of aluminum adjuvant in the presence of antigens.

In an embodiment of the invention, the following two components are kept physically separate: an aluminum-containing component including a solution of an aluminum-containing compound; and an antigen-containing component including a solution of 4 different antigens (protein bacterial toxins or toxoids) and phosphate pH 8 as a buffer and source of base to allow completion of the aluminum adjuvant precipitation reaction in the presence of the antigens. For example, in an embodiment of the invention, equal volumes, such as 6 mL, are used for each component.

The present invention provides an article of manufacture comprising the aluminum-containing component and the antigen-containing component which are kept physically separate.

The present invention also provides a method for making a vaccine comprising combining said components as well as the product of such a method. Methods for administering a vaccine to a subject (e.g., a human) comprising combining the components and injecting the vaccine into the subject (e.g., with an injection device as discussed herein) also form part of the present invention.

In an embodiment of the invention, the aluminum-containing component comprises 18.54 or 18.53 mM aluminum chloride, in water.

In an embodiment of the invention, the antigen-containing component comprises 100 mM sodium phosphate, pH 8, 9% sucrose, in water, with four types of antigens such as Antigen 1 (20 mcg/mL vpi_5mTcdA, recombinant mutant toxin from C. difficile); Antigen 2 (20 mcg/mL nap_5mTcdB, formaldehyde treated, recombinant toxoid from C. difficile); Antigen 3 (5.6 mcg/mL 3mCdtA, recombinant mutant toxin subunit from C. difficile); and, Antigen 4 (44.4 mcg/mL CdtB, toxin subunit from C. difficile).

In an embodiment of the invention, the antigen-containing component comprises one or more antigens related to C. difficile selected from the group consisting of 20 mcg/mL 5mTcdA; 20 mcg/mL 5mTcdB; 5.6 mcg/mL 3mCdtA; and 44.4 mcg/mL CdtB in a solution comprising 100 mM sodium phosphate, pH 8, and 9% sucrose.

Vaccine compositions of the present invention may include a surfactant. Surfactants that are useful in the formulations of the invention include, but are not limited to: nonionic surfactants such as polyoxyethylene sorbitan fatty acid esters (Polysorbates, sold under the trade name Tween® (Uniquema Americas LLC, Wilmington, Del.)) including Polysorbate-20 (polyoxyethylene sorbitan monolaurate), Polysorbate-40 (polyoxyethylene sorbitan monopalmitate), Polysorbate-60 (polyoxyethylene sorbitan monostearate), and Polysorbate-80 (polyoxyethylene sorbitan monooleate); polyoxyethylene alkyl ethers such as Brij® 58 (Uniquema Americas LLC, Wilmington, Del.) and Brij® 35; poloxamers (e.g., poloxamer 188); Triton® X-100 (Union Carbide Corp., Houston, Tex.) and Triton® X-1 14; NP40; Span 20, Span 40, Span 60, Span 65, Span 80 and Span 85; copolymers of ethylene and propylene glycol (e.g., the Pluronic® series of nonionic surfactants such as Pluronic® F68, Pluronic® 10R5, Pluronic® F108, Pluronic® F127, Pluronic® F38, Pluronic® L44, Pluronic® L62 (BASF Corp., Ludwigshafen, Germany); and sodium dodecyl sulfate (SDS).

In an embodiment of the invention, a composition of the present invention includes a phosphate compound; for example, a phosphate salt such as sodium phosphate or potassium phosphate.

In an embodiment of the invention, a composition of the present invention includes a base, for example, Histidine, tris(hydroxymethyl)aminomethane, HEPES, NaOH, KOH or imidazole.

In an embodiment of the invention, a composition of the present invention includes a preservative, e.g., an anti-microbial preservative, e.g., m-cresol, phenol or benzyl alcohol.

IV. Antigens

The antigens of the present invention may be any substance that is sufficiently immunogenic as to cause an immune response, against the substance, in a subject (e.g., a human) to whom the substance was administered. Antigens in the vaccines of the present invention may be lyophilized or kept in a liquid state, but physically separate from the adjuvant or adjuvant components.

Examples of antigens include a polypeptide, a nucleic acid, a polysaccharide or a conjugate of a polysaccharide to a protein, a toxoid, a subunit (e.g., of a virus), a virus like particle, a live virus, a split virus, an attenuated virus, an inactivated virus, an enveloped virus or any combination thereof.

In an embodiment of the invention, the antigen is a polysaccharide-polypeptide antigen such as a pneumococcal (e.g., S. pneumonia) polysaccharide (e.g., a cell capsule sugar)-protein (e.g., diphtheria protein) conjugate. In an embodiment of the invention, the conjugate comprises cell capture sugars of S. pneumonia conjugated to a protein (e.g., diphtheria protein), e.g., wherein the cell capsule sugars are of seven serotypes of the bacteria S. pneumoniae (4, 6B, 9V, 14, 18C, 19F and 23F), conjugated with diphtheria proteins. In an embodiment of the invention, the conjugate comprises Pneumococcal polysaccharide serotype 1, 4, 5, 6B, 7F, 9V, 14, 18C, 19F and 23F conjugated to a protein such as protein D derived from non-typeable Haemophilus influenza, tetanus toxoid carrier protein and/or diphtheria toxoid carrier protein. In an embodiment of the invention, the conjugate comprises Streptococcus pneumonia capsular polysaccharide conjugated to a diphtheria protein, e.g., Streptococcus pneumoniae type 1, 3, 4, 5, 6a, 6b, 7f, 9v, 14, 18c, 23f, 19a and 19f capsular polysaccharide conjugated to a protein such as diptheria crm197 protein. In an embodiment of the invention, one or more of the polysaccharide-protein conjugates comprising capsular polysaccharides from at least one of serotypes 1, 2, 3, 4, 5, 6A, 6B, 6C, 6D, 6E, 6G, 6H, 7F, 7A, 7B, 7C, 8, 9A, 9L, 9N, 9V, 10F, 10A, 10B, 10C, 11F, 11A, 11B, 11C, 11D, 11E, 12F, 12A, 12B, 13, 14, 15F, 15A, 15B, 15C, 16F, 16A, 17F, 17A, 18F, 18A, 18B, 18C, 19F, 19A, 19B, 19C, 20A, 20B, 21, 22F, 22A, 23F, 23A, 23B, 24F, 24A, 24B, 25F, 25A, 27, 28F, 28A, 29, 31, 32F, 32A, 33F, 33A, 33B, 33C, 33D, 33E, 34, 35F, 35A, 35B, 35C, 36, 37, 38, 39, 40, 41F, 41A, 42, 43, 44, 45, 46, 47F, 47A, 48, CWPS1, CWPS2, CWPS3 of Streptococcus pneumoniae conjugated to one or more carrier proteins.

Examples of viruses that may be used as antigens include, but not limited to, adenovirus, avian influenza, coxsackievirus, cytomegalovirus, dengue fever virus, ebola virus, Epstein-Barr virus, equine encephalitis virus, flavivirus, hepadnavirus, hepatitis A virus, hepatitis B virus, hepatitis C virus, hepatitis D virus, hepatitis E virus, herpes simplex virus, human immunodeficiency virus, human papillomavirus, influenza virus, Japanese encephalitis virus, JC virus, measles morbillivirus, marburg virus, mumps rubulavirus, orthomyxovirus, papillomavirus, parainfluenza virus, parvovirus, picornavirus, poliovirus, pox virus, rabies virus, reovirus, respiratory syncytial virus, retrovirus, rhabdovirus, rhinovirus, Rift Valley fever virus, rotavirus, rubella virus, rubeola virus, smallpox virus, togavirus, swine influenza virus, varicella-zoster virus, variola major, variola minor and/or yellow fever virus. Such viruses may be whole virus (e.g., live, attenuated or inactivated) or a subunit of such a virus.

Examples of viruses that may be used as antigens also include measles virus, mumps virus (Mumps rubulavirus), Rubella virus, varicella zoster virus or a combination of all four or three thereof (e.g., measles, mumps and rubella).

In one embodiment, an antigen is taken from one or more bacteria selected from Borrelia species, Bacillus anthracis, Borrelia burgdorferi, Bordetella pertussis, Camphylobacter jejuni, Chlamydia species, Chlamydial psittaci, Chlamydial trachomatis, Clostridium species, Clostridium tetani, Clostridium botulinum, Clostridium perfringens, Corynebacterium diphtherias, Coxiella species, an Enterococcus species, Erlichia species, Escherichia coli, Francisella tularensis, Haemophilus species, Haemophilus influenzae, Haemophilus parainfluenzae, Lactobacillus species, a Legionella species, Legionella pneumophila, Leptospirosis interrogans, Listeria species, Listeria monocytogenes, Mycobacterium species, Mycobacterium tuberculosis, Mycobacterium leprae, Mycoplasma species, Mycoplasma pneumoniae, Neisseria species, Neisseria meningitidis, Neisseria gonorrhoeae, Pneumococcus species, Pseudomonas species, Pseudomonas aeruginosa, Salmonella species, Salmonella typhi, Salmonella enterica, Streptococcus species, Rickettsia species, Rickettsia ricketsii, Rickettsia typhi, Shigella species, Staphylococcus species, Staphylococcus aureus, Streptococcus species, Streptococccus pneumoniae, Streptococcus pyrogenes, Streptococcus mutans, Treponema species, Treponema pallidum, a Vibrio species, Vibrio cholerae and Yersinia pestis. Such bacteria may be a whole cell (e.g., live, attenuated or inactivated) or a polypeptide or polysaccharide of such a bacterium.

In one embodiment, an antigen is taken from one or more fungus selected from an Aspergillus species, Candida species, Candida albicans, Candida tropicalis, Cryptococcus species, Cryptococcus neoformans, Entamoeba histolytica, Histoplasma capsulatum, Leishmania species, Nocardia asteroides, Plasmodium falciparum, Toxoplasma gondii, Trichomonas vaginalis, Toxoplasma species, Trypanosoma brucei, Schistosoma mansoni, Fusarium species, and/or Trichophyton species. Such fungi may be a whole cell (e.g., live, attenuated or inactivated) or a polypeptide or polysaccharide of such a fungus.

In one embodiment, an antigen is taken from one or more parasites selected from Plasmodium species, Toxoplasma species, Entamoeba species, Babesia species, Trypanosoma species, Leshmania species, Pneumocystis species, Trichomonas species, Giardia species, and/or Schisostoma species. Such parasites may be a whole cell (e.g., live, attenuated or inactivated) or a polypeptide or polysaccharide of such a parasite.

In an embodiment of the invention, the active ingredient is a nucleic acid. For example, in an embodiment of the invention, the nucleic acid is selected form DNA, RNA, SiRNA, mRNA, etc.

In an embodiment of the invention, the active ingredient is a toxoid. For example, in an embodiment of the invention, the toxoid is diphtheria toxoid or tetanus toxoid or toxoids from C. difficile.

In an embodiment of the invention, the active ingredient is a subunit of a virus. For example, in an embodiment of the invention, the viral subunit is selected from influenza flu virus, Herpes Simplex Virus, Respiratory Synctial Virus F protein, HIV, Glycoprotein from VSV etc.

In an embodiment of the invention, the active ingredient is a virus like particle (VLP). For example, in an embodiment of the invention, the VLP is a virus-like particle of a human papillomavirus or cytomegalovirus.

In an embodiment of the invention, the active ingredient is a split virus. For example, in an embodiment of the invention, the split virus is influenza virus.

In an embodiment of the invention, the antigen is an anti-cancer antigen which may be used in an anti-cancer vaccine. For example, the antigen can be a tunmor-associated antigen (TAA) such as gp100, Tecemotide, Folate-binding protein (FBP), AE37, GP2, E75 (nelipepimut-S), HER2/neu or rindopepimut. In an embodiment of the invention, the anti-cancer antigen is conjugated to a virus.

V. Drying

The present invention includes vaccines wherein one or more components, before reconstitution, are dried, e.g., lyophilized. A composition is dried if moisture is removed yielding a solid, substantially lacking liquid, such as water. Dried compositions may be, for example, lyophilized, microwave vacuum dried (e.g., of a lyosphere), spray-dried, pulse combustion dried or drum dried. See e.g., Encyclopedia of Agriculture, Food, and Biological Engineering. Marcel Dekker, Inc or Xu & Sunada, Chem Pharm Bull (Tokyo) 55(11):1545-50 (2007). The lyophilized formulation components may be in the form of a particle, e.g., a pellet, bead or sphere of lyophilized material. See e.g., A. S. Mujumdar (2007). Handbook of Industrial Drying. CRC Press.

In an embodiment of the invention, lyophilization or drying is done by a method which excludes direct contact of liquid nitrogen to a droplet containing a vaccine component such as a pre-formed aluminum adjuvant.

In an embodiment of the invention, a formulation component is lyophilized by freezing and subjecting the frozen composition to low pressure so as to cause water in the component to sublimate. For example, in an embodiment of the invention, the component is subjected to a three step lyophilization process: pre-freezing, primary drying and secondary drying as follows.

Pre-freezing in which the component is first cooled to below its eutectic temperature (e.g., −50° C. to −105° C. or less).

Primary drying in which the frozen component is then subjected to low pressure (e.g., a vacuum or partial vacuum, e.g., about 1, 2 or 3 millibars of pressure) causing water in the sample to sublimate. When subjected to the low temperature and low pressure, the process is continued until the component appears as a dried matrix.

Secondary drying in which after primary drying is complete, and all ice has sublimed, bound moisture may still be present in the product. Residual water is removed at a warmer temperature. This process is called isothermal desorption as the bound water is desorbed from the product.

Lyospheres can be made, for example, by loading an aliquot of liquid in the form of a droplet (e.g., about 20, 50, 100 or 250 microliters) onto a solid, flat surface in such a way that the droplet remains intact. In an embodiment of the invention, the surface is a plate, e.g., a metal plate, e.g. at a temperature of about −180° C. to about −196° C. or about −180° C. to about −273° C. For example, in an embodiment of the invention, the liquid is loaded onto the surface by way of a dispensing tip. In an embodiment of the invention, the liquid is dispensed at a dispensing speed of about 3 ml/min to about 75 ml/min, about 5 ml/min to about 75 ml/min; about 3 ml/min to about 60 ml/min, about 20 ml/min to about 75 ml/min; and about 20 ml/min to about 60 ml/min. In an embodiment of the invention, the aliquot that is dispensed is 250 microliters and the dispensing speed is between about 5 ml/min to about 75 ml/min, or wherein the aliquot is 100 microliters and the dispensing speed is between about 3 ml/min to about 60 ml/min. In an embodiment of the invention, the gap between a dispensing tip and the surface onto which the liquid is dispensed if about 0.1 cm or more (e.g., about 0.5 cm or between 0.1 cm and 1 cm or between 0.1 cm and 0.75 cm). Once on the surface, the droplet is frozen and then subjected to drying. Methods for making lyospheres are known in the art. See e.g., U.S. Pat. No. 5,656,597; WO2013066769; WO2014093206; WO2015057540; WO2015057541 or WO2015057548.

In an embodiment of the invention, the formulation component is lyophilized by microwave vacuum-drying. Microwave vacuum-drying is a drying method done under reduced pressure, where the boiling point of water and the oxygen content of the atmosphere are lower. For example, in an embodiment of the invention, a formulation component are made by dispensing an aliquot of liquid as a single droplet onto a solid surface, wherein the temperature of the surface is at about −90° C. or below, in a manner that maintains droplet as a single droplet as it contacts and freezes on the surface as a frozen pellet; and applying microwave radiation to the frozen pellet under a pressure below atmospheric pressure to produce a dried pellet, such as a sphere. See, e.g., U.S. Pat. Nos. 4,389,794; 4,664,924; 4,809,596; 4,882,851. In an embodiment of the invention, a component of a formulation is spray-dried.

Spray drying is the process of converting a liquid mixture or solution to a powder. This is done by removing the moisture component from the liquid. The liquid is sprayed, e.g., through a nozzle, into a chamber that simultaneously has hot air being blown into it. As droplets of the solution are released through the nozzle and come in contact with the hot air, the moisture content of each droplet is removed, thus turning it from liquid to powder form.

VI. Method of Treatment

The present invention provides methods for administering a vaccine to a subject in need thereof comprising reconstituting the vaccine formulation of the present invention and introducing the formulation to the subject (e.g., injecting the formulation). The present invention also includes methods for treating or preventing infectious diseases or cancer or for immunizing/vaccinating a subject against an antigen by administering a reconstituted vaccine formulation of the present invention. In an embodiment of the invention, the infectious disease is a bacterial, viral, fungal and/or parasitic infection.

The term “administer” with regard to a vaccine formulation of the present invention refers to the introducing of the formulation of the present invention in the body of a subject, e.g., intramuscular injection, subcutaneous injection, intravenous injection, intradermal injection, oral introduction, intranasal introduction.

In an embodiment of the invention, the methods include receiving instructions, from a clinician to a subject, to self-administer the vaccine formulation and, in response to the clinician's instructions, reconstituting the formulation and self-administering the formulation to the subject's own body. In an embodiment of the invention, the method includes reconstitution of the vaccine formulation and administration into the body of a subject, e.g., administration by a clinician to a subject.

In an embodiment of the invention, the viral infection is with a virus selected from adenovirus, avian influenza, coxsackievirus, cytomegalovirus, dengue fever virus, ebola virus, Epstein-Barr virus, equine encephalitis virus, flavivirus, hepadnavirus, hepatitis A virus, hepatitis B virus, hepatitis C virus, hepatitis D virus, hepatitis E virus, herpes simplex virus, human immunodeficiency virus, human papillomavirus, influenza virus, Japanese encephalitis virus, JC virus, measles morbillivirus, marburg virus, mumps rubulavirus, orthomyxovirus, papillomavirus, parainfluenza virus, parvovirus, picornavirus, poliovirus, pox virus, rabies virus, reovirus, respiratory syncytial virus, retrovirus, rhabdovirus, rhinovirus, Rift Valley fever virus, rotavirus, rubella virus, rubeola virus, smallpox virus, togavirus, swine influenza virus, varicella-zoster virus, variola major, variola minor, and yellow fever virus.

For example, in an embodiment of the invention, the bacterial infection is with a bacterium selected from Borrelia species, Bacillus anthracis, Borrelia burgdorferi, Bordetella pertussis, Camphylobacter jejuni, Chlamydia species, Chlamydial psittaci, Chlamydial trachomatis, Clostridium species, Clostridium tetani, Clostridium botulinum, Clostridium perfringens, Corynebacterium diphtherias, Coxiella species, an Enterococcus species, Erlichia species, Escherichia coli, Francisella tularensis, Haemophilus species, Haemophilus influenzae, Haemophilus parainfluenzae, Lactobacillus species, a Legionella species, Legionella pneumophila, Leptospirosis interrogans, Listeria species, Listeria monocytogenes, Mycobacterium species, Mycobacterium tuberculosis, Mycobacterium leprae, Mycoplasma species, Mycoplasma pneumoniae, Neisseria species, Neisseria meningitidis, Neisseria gonorrhoeae, Pneumococcus species, Pseudomonas species, Pseudomonas aeruginosa, Salmonella species, Salmonella typhi, Salmonella enterica, Streptococcus species, Rickettsia species, Rickettsia ricketsii, Rickettsia typhi, Shigella species, Staphylococcus species, Staphylococcus aureus, Streptococcus species, Streptococccus pneumoniae, Streptococcus pyrogenes, Streptococcus mutans, Treponema species, Treponema pallidum, a Vibrio species, Vibrio cholerae and Yersinia pestis.

For example, in an embodiment of the invention, the fungal infection is with a fungus selected from an Aspergillus species, Candida species, Candida albicans, Candida tropicalis, Cryptococcus species, Cryptococcus neoformans, Entamoeba histolytica, Histoplasma capsulatum, Leishmania species, Nocardia asteroides, Plasmodium falciparum, Toxoplasma gondii, Trichomonas vaginalis, Toxoplasma species, Trypanosoma brucei, Schistosoma mansoni, Fusarium species and Trichophyton species.

For example, in an embodiment of the invention, the parasitic infection is with a parasite selected from Plasmodium species, Toxoplasma species, Entamoeba species, Babesia species, Trypanosoma species, Leshmania species, Pneumocystis species, Trichomonas species, Giardia species and Schisostoma species.

In an embodiment of the invention, the cancer is melanoma, lung cancer, colorectal cancer, ovarian cancer, liver cancer, prostate cancer or breast cancer.

In an embodiment of the invention, a vaccine is in association with an immune stimulant such as a cytokine, GM-CSF (granulocyte-macrophage colony-stimulating factor), IL2, IL5 or IL10.

The term “in association with” indicates that the components, a vaccine composition along with another agent such as an immune stimulant can be formulated into a single composition, e.g., for simultaneous delivery, or formulated separately into two or more compositions (e.g., a kit). Each component can be administered to a subject at a different time than when the other component is administered; for example, each administration may be given non-simultaneously (e.g., separately or sequentially) at intervals over a given period of time. Moreover, the separate components may be administered to a subject by the same or by a different route. If the other component is formulated as part of the vaccine composition, then, in an embodiment of the invention, it is kept physically separate from the other composition components until reconstitution.

The following examples are intended to promote a further understanding of the present invention.

### Example 1

This example shows preparation of aluminum adjuvant lyobeads from pre-made aluminum adjuvant, and reconstitution without significant freezing or thawing induced agglomeration.

The following components were used to form the vaccine discussed in this example:

(1) Pre-formed dried aluminum adjuvant (example: lyobeads) reconstituted with
(2) Liquid (see FIG. 1), comprising:
Water
Optionally salt(s), buffer(s), surfactant(s) and/or excipients
Optionally antigen(s)
Optionally additional adjuvant(s) Optionally preservative(s)

### Embodiments in the Instant Example

(1) Lyobeads of MAPA (aluminum phosphate adjuvant) with pre-lyo composition of Formulation 3 or Formulation 2.

TABLE 1
Concentrations before lyophilization.

| Component | Formulation 3 | Formulation 2 |
|---|---|---|
| MAPA | 625 mcg Al/mL | 1250 mcg Al/mL |
| trehalose | 23% (w/v) | 16% (w/v) |
| Sodium Chloride | 35 mM | 70 mM |
| Histidine (base) | 0.8 mM | 0.5 mM |

Three different formulations of aluminum adjuvant were prepared.

### Formulation 1

1250 mcg Al/mL aluminum adjuvant
150 mM sodium chloride

### Formulation 2

1250 mcg Al/mL aluminum adjuvant
16% (w/v) trehalose
70 mM sodium chloride
0.5 mM histidine base

### Formulation 3

625 mcg Al/mL aluminum adjuvant
23% (w/v) trehalose
35 mM sodium chloride
0.8 mM histidine base

Formulations #2 and #3 were prepared as lyospheres. Briefly, the suspensions were mixed, filled into deep-well multichannel pipette plates, and 50 μL aliquots of each suspension were dropped onto an ultracold metal surface (using the “CRYOMEK”) resulting in very fast freezing of 50 μL droplets. (A similar process was also done by hand using a pipette and dropping 100 μL aliquots onto a liquid nitrogen cooled metal plate.) These beads were kept frozen (target −70° C. or below) until they were lyophilized. Lyophilization was performed in a LYOSTAR II unit with a suitable lyophilization cycle.

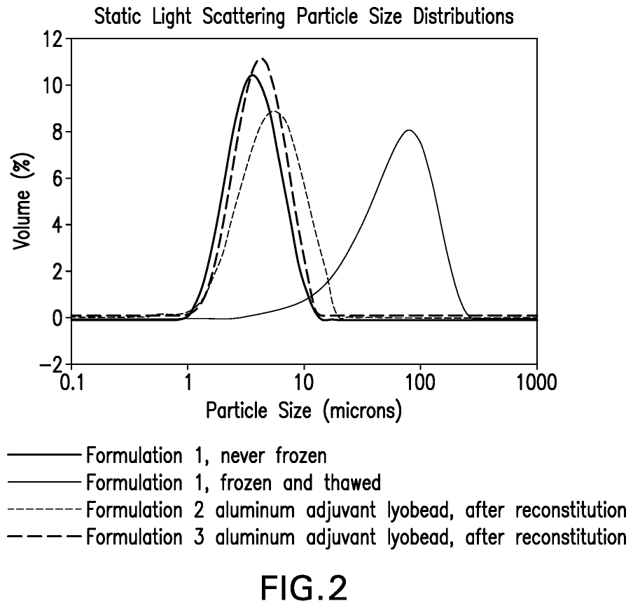

Freezing and/or thawing induced agglomeration is generally interpreted as evidence of damage to a vaccine or aluminum adjuvant. As shown in FIG. 2, mechanically size-reduced adjuvant in Formulation #1 that has never been frozen had the expected size distribution as measured by static light scattering. Adjuvant in Formulation #1 that was allowed to settle, freeze solid and be thawed experienced significant agglomeration. Formulations #2 and #3, after being frozen as beads, lyophilized, and reconstituted with water, had a size distribution that was consistent with adjuvant in Formulation #1 that was never frozen. The reconstituted lyobeads had a very different size distribution compared to Formulation #1 which had been frozen and thawed. The reconstituted lyobeads did not show evidence of agglomeration, even while having experienced freezing and storage as a frozen liquid and as a frozen dried bead. The dried beads remained physically stable for hours, days, weeks and longer at room temperature and above. Surprisingly, a particle size difference was noted between reconstituted lyobeads from formulation #2 and #3. Formulation #3 particle sizes appeared slightly smaller. Without desiring to be constrained by theory, this might be due to the lower concentration of aluminum adjuvant, higher concentration of trehalose, lower concentration of sodium chloride, higher concentration of histidine base, and/or difference in pH.

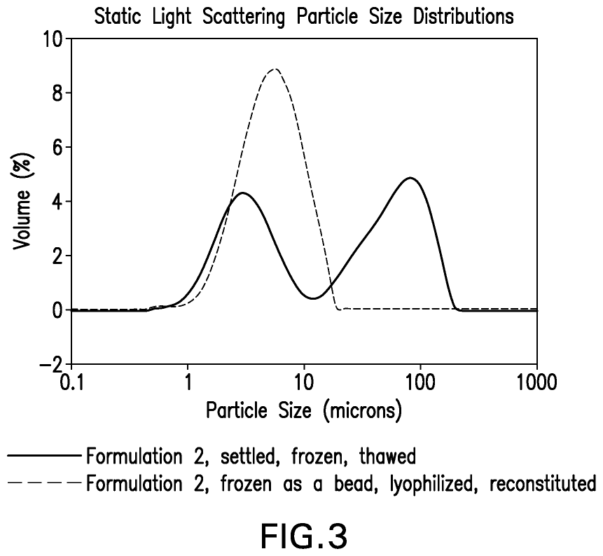

For Formulation 2, a comparison was made between allowing the aluminum adjuvant suspension in high trehalose to settle, be frozen, then thawed, versus freezing as a lyobead (dispensing a droplet of resuspended suspension on an ultracold plate), lyophilizing, and reconstituting. Based on static light scattering measurements (FIG. 3), while there did seem to be some freeze-thaw protection likely attributable to the high trehalose concentration, the lyobead process for the same formulation showed smaller particle size and was much better protected from agglomeration.

### Example 2

This example shows preparation of vaccines by co-reconstitution of aluminum adjuvant lyobeads (prepared from pre-made aluminum adjuvant), and antigen lyobeads, without significant freezing or thawing induced agglomeration.

The following components were used to form the vaccine discussed in this example:

(1) Pre-formed dried aluminum adjuvant (example: lyobeads)

### (2) Formulated Antigen(s);

reconstituted with
(3) Liquid, comprising:
Water
Optionally salt(s), buffer(s), surfactant(s) and/or excipients
Optionally antigen(s)
Optionally additional adjuvant(s)
Optionally preservative(s)

### See FIG. 4.

### Embodiments in the Instant Example

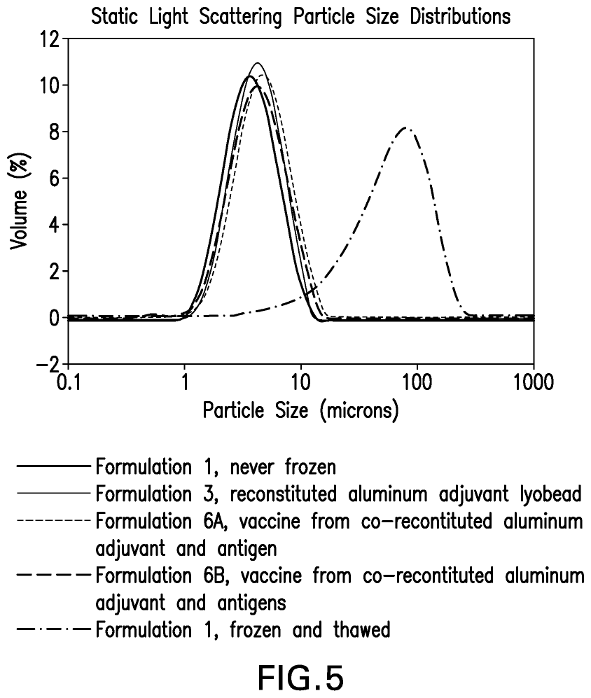

(1) Lyobeads of MAPA with pre-lyo composition of Formulation 3 or Formulation 2
(2) PCV antigen formulations, including 15-valent (data in FIG. 5), 1-valent Type 3 (data not shown), 1-valent Type 19A (data in FIG. 5) pneumococcal polysaccharide protein conjugate antigens comprising formulations before drying:
320 mcg PnPs/mL CRM-PnPs (pneumococcal polysaccharide) CRM-PnPs conjugate antigen(s)
100 mM histidine
20% (w/v) trehalose
50 mM sodium chloride
0.1% polysorbate 20
pH 6-7.5

Vaccines with one antigen (“mono-valent”) or more than one antigen (“multi-valent”, or “combination”) were prepared by co-packaging dried lyobeads of aluminum adjuvant (Formulation #3) with lyobeads of formulated antigen or antigens. Water was added to the dried beads to reconstitute aluminum-adjuvanted vaccines.

### Formulation 1

1250 mcg Al/mL aluminum adjuvant
150 mM sodium chloride
   Formulation 3 (lyobeads of aluminum adjuvant)
625 mcg Al/mL aluminum adjuvant
23% (w/v) trehalose
35 mM sodium chloride
0.8 mM histidine base
   Formulation 4 (for lyobeads comprising an antigen)
20% (w/v) trehalose
50 mM histidine (base)
50 mM histidine hydrochloride
320 mcg PnPs/mL CRM-PnPs (Pneumococcal polysaccharide) conjugate antigen, type 19A
50 mM sodium chloride
0.1% polysorbate 20
pH 6
   Formulation 5 (for lyobeads comprising multiple different antigens)
20% (w/v) trehalose
100 mM histidine (base)
320 mcg PnPs/mL CRM-PnPs (Pneumococcal polysaccharide) conjugate antigen, a blend of 15 conjugates with different types of polysaccharides
50 mM sodium chloride
0.1% polysorbate 20
pH 7.5
   Formulation 6A or 6B (formulations after reconstitution of lyobeads of Formulation 3+Formulation 4, or Formulation 3+Formulation 5)
13.2% (w/v) trehalose
250 mcg Al/mL aluminum adjuvant
64 mcg PnPs/mL CRM-PnPs conjugate antigen, all type 19A (A) or a blend comprising 15 PnPs types (B)
24 mM sodium chloride
20.3 mM histidine (base)
0.02% polysorbate 20

Static light scattering (SLS) measurements were made to compare the particle size distribution of never frozen and frozen/thawed aluminum adjuvant, and lyobeads of aluminum adjuvant after reconstitution, with vaccines (aluminum adjuvant+one or more antigens) prepared from co-packaging aluminum adjuvant lyobeads with antigen lyobeads and reconstituting together (FIG. 5). Vaccines prepared in this manner showed particle size distributions similar to never frozen aluminum adjuvant, and did not show evidence of freeze or thaw induced agglomeration.

### Example 3

This example shows preparation of aluminum adjuvant by reconstitution of co-packaged but separate lyobeads of aluminum containing solution and phosphate/base containing solution which react to form aluminum adjuvant in a vial (or in a reconstitution chamber).

The following components were used to form the vaccine discussed in this example:

(1) Dried formulation comprising some of the reactants needed to form aluminum adjuvant (for example, an aluminum containing compound)
(2) Dried formulation comprising others of the reactants needed to form aluminum adjuvant (for example, a phosphate containing compound and/or a base) reconstituted with
(3) Liquid, comprising:
Water
Optionally salt(s), buffer(s), surfactant(s) and/or excipients
Optionally antigen(s)
Optionally additional adjuvant(s)
Optionally preservative(s)

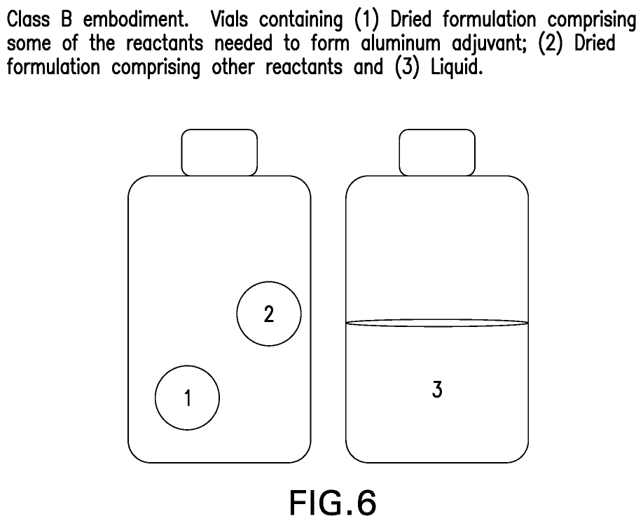

### See FIG. 6.

### Embodiments in the Instant Example

(Concentrations before lyophilization)
(1) 92.66 mM aluminum chloride, 25% (w/v) trehalose;
(2) 92.66 mM sodium phosphate tribasic, 25% (w/v) trehalose;
(2) 92.47 mM sodium phosphate tribasic, 24.95% (w/v) trehalose; or
(2) 92.29 mM sodium phosphate tribasic, 24.9% (w/v) trehalose

Aluminum adjuvant can be prepared by the reaction of an aluminum compound containing solution with a phosphate containing solution and/or a base-providing solution, when enough base is provided for the precipitation reaction to occur. Aluminum and phosphate containing solutions are not particulate suspensions and do not experience the freeze-thaw induced agglomeration that can damaged aluminum adjuvant. By delaying formation of the aluminum adjuvant until it is created in a vial or delivery device, and in some cases immediately before injection, manufacturing and shipping conveniences can be achieved including avoiding freeze-thaw induced adjuvant agglomeration.

One solution of an aluminum-containing compound, and three solutions of a phosphate/base-containing compound were prepared.

### Formulation 1

1250 mcg Al/mL aluminum adjuvant
150 mM sodium chloride

### Formulation 2

1250 mcg Al/mL aluminum adjuvant
16% (w/v) trehalose
70 mM sodium chloride
0.5 mM histidine base
   Formulation 7 (an aluminum containing compound)
92.66 mM aluminum chloride
25% (w/v) trehalose
   Formulation 8A (a phosphate/base containing compound)
92.66 mM sodium phosphate tribasic
25% (w/v) trehalose
   Formulation 8B (a phosphate/base containing compound)
92.47 mM sodium phosphate tribasic
24.95% (w/v) trehalose
10 mM sodium hydroxide
   Formulation 8C (a phosphate/base containing compound)
92.29 mM sodium phosphate tribasic
24.9% (w/v) trehalose
20 mM sodium hydroxide

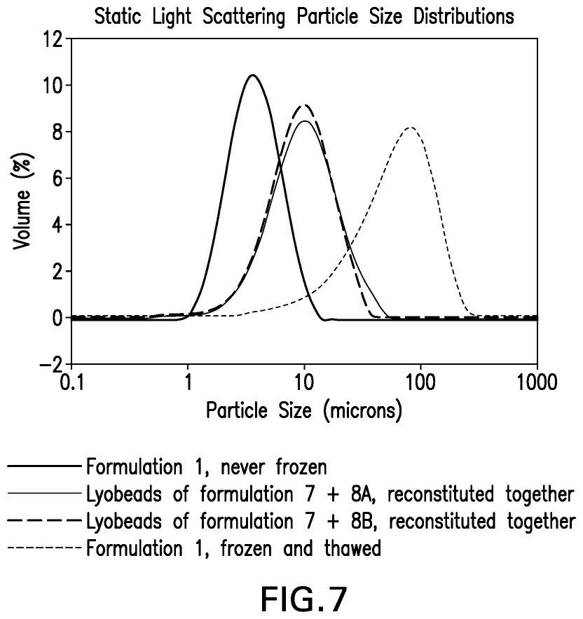

Frozen and lyophilized beads of Formulation 7 were co-packaged with frozen and lyophilized beads of Formulation 8 (A, B, or C). Addition of water to reconstitute the lyobeads initiated the aluminum adjuvant precipitation reaction, which occurred rapidly. The particle size distribution of the resulting aluminum adjuvant suspension was measured by static light scattering and compared to mechanically size-reduced aluminum adjuvant (Formulation 1), never frozen and frozen/thawed. The particle size appeared larger than mechanically size-reduced aluminum adjuvant in saline that had not been frozen, but significantly smaller than the same aluminum adjuvant agglomerated by freeze-thaw (FIG. 7). Despite the size increase over mechanically size-reduced aluminum adjuvant in saline, in-vial or in-device reconstitution leading to reaction to form aluminum adjuvant could represent a significant improvement in manufacturing and shipping flexibility, and quality over freeze-thaw agglomerated aluminum adjuvant, and may prove acceptable and efficacious in vaccines with additional testing.

### Example 4

This examples shows preparation of vaccines (aluminum adjuvant+antigen or antigens) by reconstitution of co-packaged but separate lyobeads of aluminum containing solution and phosphate/base containing solution which react to form aluminum adjuvant in a vial (or in a reconstitution chamber), and using that newly formed aluminum adjuvant suspension to reconstitute antigen lyobeads to form a complete vaccine.

The following components were used to form the vaccine discussed in this example:

(1) Dried formulation comprising some of the reactants needed to form aluminum adjuvant (for example, an aluminum containing compound)
(2) Dried formulation comprising others of the reactants needed to form aluminum adjuvant (for example, a phosphate containing compound and/or a base) reconstituted with
(3) Liquid, comprising:
Water
Optionally salt(s), buffer(s), surfactant(s) and/or excipients
Optionally antigen(s)
Optionally additional adjuvant(s)
Optionally preservative(s)
   using the reconstituted, newly formed aluminum adjuvant to further reconstitute (4) formulated Antigen(s)

### See FIG. 8.

### Embodiments in the Instant Example

(1) 92.66 mM aluminum chloride, 25% (w/v) (before drying)
(2) 92.66 mM sodium phosphate tribasic, 25% (w/v) trehalose (before drying)
(4) 15-valent antigen, formulation before drying:
320 mcg PnPs/mL CRM-PnPs (pneumococcal polysaccharide) conjugate antigens
50 mM histidine base
50 mM histidine hydrochloride
20% (w/v) trehalose
50 mM sodium chloride
0.1% polysorbate 20
pH 6

Monovalent (one antigen type) and multivalent (combinations of more than one antigen type) were prepared by multiple methods.

### Method 1

Prepare lyobeads of pre-formed aluminum adjuvant (mechanically size-reduced)
Prepare lyobeads of formulated antigen or antigen combinations
Co-package the lyobeads of aluminum adjuvant and antigen
Reconstitute together

### Method 2

Prepare lyobeads of an aluminum-containing compound
Prepare lyobeads of a phosphate/base-containing compound
Prepare lyobeads of formulated antigen or antigen combinations
Co-package the lyobeads of aluminum and phosphate/base containing compounds
Reconstitute to allow reaction to form aluminum adjuvant
Use the aluminum adjuvant to reconstitute the antigens lyobead

Comparisons of particle size distribution were made by static light scattering measurements. Relevant formulations for comparisons in this example included the following:

### Formulation 1

1250 mcg Al/mL aluminum adjuvant
150 mM sodium chloride

### Formulation 3

625 mcg Al/mL aluminum adjuvant
23% (w/v) trehalose
35 mM sodium chloride
0.8 mM histidine base
   Formulation 7 (an aluminum containing compound)
92.66 mM aluminum chloride
25% (w/v) trehalose
   Formulation 8A (a phosphate/base containing compound)
92.66 mM sodium phosphate tribasic
25% (w/v) trehalose
   Formulation 9 (for lyobeads comprising multiple different antigens)
20% (w/v) trehalose
50 mM histidine (base)
50 mM histidine hydrochloride
320 mcg PnPs/mL CRM-PnPs (pneumococcal polysaccharide) CRM-PnPs conjugate antigens
50 mM sodium chloride
0.1% polysorbate 20
pH 6

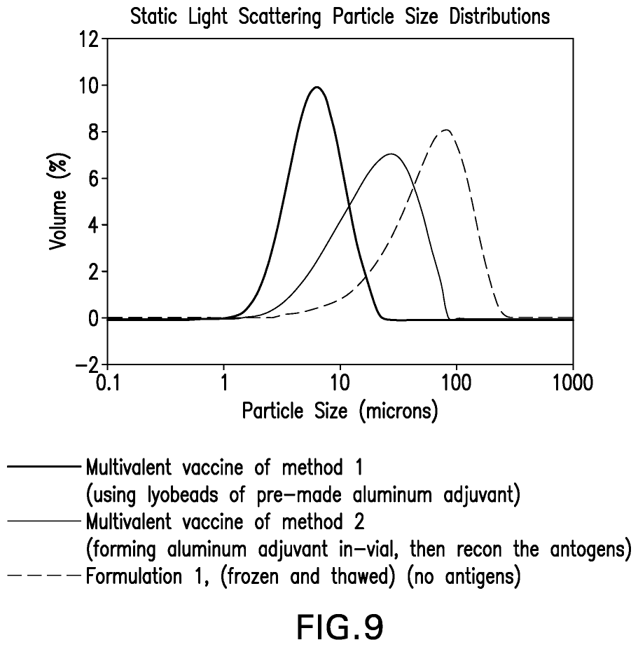

Frozen aluminum adjuvant in saline (Formulation 1, frozen and thawed) is generally accepted to be damaged by freeze-thaw agglomeration. Multivalent vaccine prepared by Method 1 (lyobeads of pre-made mechanically size reduced aluminum adjuvant) resulted in the smallest particle size adjuvant FIG. 9). Multivalent vaccine prepared by Method 2 (lyobeads of aluminum and phosphate/base containing compounds which react upon co-reconstitution to form aluminum adjuvant) had a larger particle size, but still significantly smaller than freeze-thaw agglomerated aluminum adjuvant without antigen. The precise nature of the larger particle size vaccine resulting from Method 2 is not yet fully understood, but it is not a result of freeze-thaw agglomeration. Despite the size increase over a vaccine prepared using reconstituted lyobeads of mechanically size-reduced aluminum adjuvant, in-vial or in-device reconstitution of aluminum and phosphate/base solutions leading to reaction to form aluminum adjuvant, followed by reconstitution of a lyophilized antigen with that newly formed adjuvant, could avoid the risks of freeze-thaw damage present in many current vaccines and represent a significant improvement in manufacturing and shipping flexibility. With additional testing, it may prove acceptable and efficacious in vaccines.

### Example 5

This example shows preparation of monovalent and multivalent HPV vaccines by combining a liquid solution of aluminum- and phosphate-containing compounds (that has not yet reacted to form an aluminum adjuvant suspension) with formulated antigen in either the liquid or lyophilized state, resulting in reaction to form aluminum adjuvant in the presence of one or more antigens.

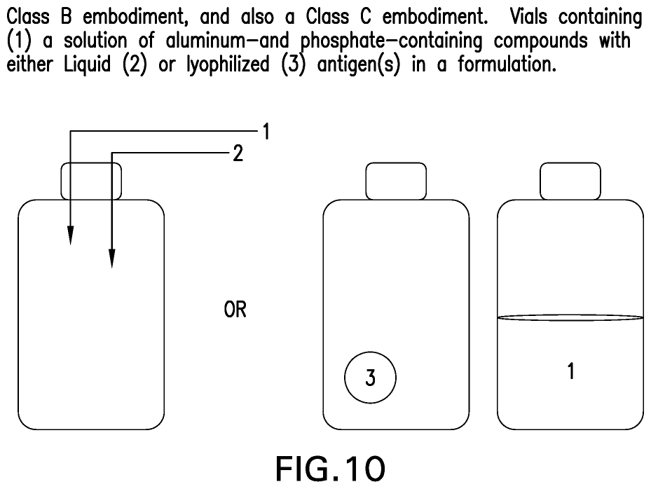

Combining (1) a solution of aluminum- and phosphate-containing compounds (lacking sufficient base to complete the reaction to form aluminum adjuvant suspension) with either liquid (2) or lyophilized (3) antigen(s) in a formulation, the formulated antigen(s) comprising a buffer, salt, and/or base allows completion of the aluminum adjuvant precipitation reaction in the presence of antigen(s). See FIG. 10.

### Embodiments in the Instant Example

(1) the solution obtained by mixing equal volumes of 37.08 mM aluminum chloride in water with 33.38 mM sodium phosphate pH 7.0 in water, resulting in 18.54 mM aluminum chloride, 16.69 mM sodium phosphate, at acidic pH. For some of the comparisons in Example 5, this is further diluted 1:1 with water.
(2) (50, 80, or 75) mM sodium phosphate dibasic, (50, 20, or 25) mM sodium phosphate monobasic, 160 mcg/mL of HPV VLP type 16, 5 mM histidine, 125 mM sodium chloride, 0.005% polysorbate 80, in water.
(3) (20 or 42.5) mM histidine base, 4.5% sucrose, HPV VLP types 6 (40 mcg/mL), 11 (80 mcg/mL), 16 (80 mcg/mL), and 18 (40 mcg/mL), 125 mM sodium chloride, 0.005% polysorbate 80, lyophilized.

A solution of aluminum- and phosphate-containing compounds (lacking sufficient base to complete the reaction to form aluminum adjuvant suspension) was mixed with formulated HPV virus-like particles of a single VLP type (a liquid formulation) or multiple VLP types (a lyophilized formulation). Buffers present with the formulated antigens provided the base that was lacking to allow reaction to form aluminum adjuvant in the presence of the antigens. These reactions were performed at small volumes (for example 1 mL added to 1 mL) as could be the case in vaccine vials or delivery devices. Assays were performed to characterize the antigens and vaccines before and after the aluminum adjuvant formation reaction. These included pH, virus-like particle average size by dynamic light scattering (after aluminum adjuvant dissolution), and virus-like particle relative potency by Biacore (after aluminum adjuvant dissolution). With further testing, HPV vaccines made by formation of the aluminum adjuvant in the presence of antigens, in a vial or delivery device may prove acceptable and efficacious.

### Formulation 11

18.54 mM aluminum chloride hexahydrate
16.69 mM sodium phosphate
In water, at acidic pH

### Formulation 17

50 mM sodium phosphate dibasic
50 mM sodium phosphate monobasic
160 mcg/mL of HPV VLP type 16
5 mM histidine
125 mM sodium chloride
0.005% polysorbate 80
In water

### Formulation 18

80 mM sodium phosphate dibasic
20 mM sodium phosphate monobasic
160 mcg/mL of HPV VLP type 16
5 mM histidine
125 mM sodium chloride
0.005% polysorbate 80
In water

### Formulation 19

75 mM sodium phosphate dibasic
25 mM sodium phosphate monobasic
160 mcg/mL of HPV VLP type 16
5 mM histidine
125 mM sodium chloride
0.005% polysorbate 80
In water

### Formulation 20

20 mM histidine base
4.5% sucrose
40 mcg/mL of HPV VLP type 6
80 mcg/mL of HPV VLP type 11
80 mcg/mL of HPV VLP type 16
40 mcg/mL of HPV VLP type 18
125 mM sodium chloride
0.005% polysorbate 80
lyophilized
   Formulation 21
42.5 mM histidine base
4.5% sucrose
40 mcg/mL of HPV VLP type 6
80 mcg/mL of HPV VLP type 11
80 mcg/mL of HPV VLP type 16
40 mcg/mL of HPV VLP type 18
125 mM sodium chloride
0.005% polysorbate 80
lyophilized

### Formulation 22

9.27 mM aluminum chloride hexahydrate
8.34 mM sodium phosphate
In water, at acidic pH

TABLE 2
Relative antigenicity of various formulations

| | Aluminum | | | Relative antigenicity |
|---|---|---|---|---|
| | adjuvant | | DLS Z-Ave | (% response versus a |
| Antigen solution or | concentration | | (nm) particle | control in Biacore |
| Vaccine evaluated | (mcg Al/mL) | pH | size | assay) |
| Form. 17 alone | 0 | 6.61 | 64 | 97 |
| Form 17 + Form 11 | 250 | 5.82 | 65 | 90 |
| (1:1 volumes) | | | | |
| Form. 18 alone | 0 | 7.15 | 64 | 99 |
| Form 18 + Form 11 | 250 | 6.47 | 64 | 93 |
| (1:1 volumes) | | | | |
| Form. 19 alone | 0 | 7.01 | 64 | 102 |
| Form 19 + Form 11 | 250 | 6.37 | 66 | 93 |
| (1:1 volumes) | | | | |
| Form. 20, | 0 | 6.87 | 112 | 112 |
| reconstituted with | | | | |
| water | | | | |
| Form. 20, | 240 | 5.02 | 92 | 89 |
| reconstituted with | | | | |
| Form. 22 | | | | |
| Form. 21, | 0 | 7.19 | 106 | 110 |
| reconstituted with | | | | |
| water | | | | |
| Form. 21, | 240 | 6.06 | 91 | 89 |
| reconstituted with | | | | |
| Form. 22 | | | | |
| Form. 21, | 0 | 7.19 | 106 | 110 |
| reconstituted with | | | | |
| water | | | | |
| Form. 21, | 478 | 5.37 | 94 | 84 |
| reconstituted with | | | | |
| Form. 11 | | | | |

### Example 6

This example shows preparation of aluminum adjuvant by combination of a liquid solution comprising aluminum-containing compounds and phosphate-containing compounds (said solution does not by itself react to form an aluminum adjuvant suspension with precipitated solids), with a lyophilized or spray dried formulation which provides the base needed to complete the aluminum adjuvant formation reaction.

The following components were used to form the vaccine discussed in this example:

(1) Dried formulation comprising some of the reactants needed to form aluminum adjuvant (for example, a formulated antigen with phosphate and/or buffer and/or base, optionally buffer and/or excipients, optionally additional adjuvants, optionally preservative(s)) reconstituted with:
(2) Liquid, comprising:
Water
Others of the reactants needed to form aluminum adjuvant (for example, an aluminum-containing compound and a phosphate-containing compound, lacking sufficient base for completion of the aluminum adjuvant precipitation reaction)
Optionally buffer(s), salt(s), surfactant(s) and/or excipient(s)
Optionally additional adjuvant(s)
Optionally preservative(s)

### Embodiments in the Instant Example

(1) Spray dried formulations comprising (before spray drying): 1% sucrose, 9% mannitol, 50 mM phosphate, pH 7.4 or pH 7.8; or (Alternate 1) In-vial lyophilized formulations comprising (before lyophilization) 6% sucrose, 100 mM histidine, pH 6.0 or 7.0 or 7.4
(2) 10.49 mM aluminum chloride, 9.44 mM sodium phosphate, in water at acidic pH

Lyophilized and spray dried formulations were prepared. Addition of a solution of aluminum- and phosphate-containing compounds (which is not the same thing as an aluminum adjuvant suspension) to the lyophilized or spray dried formulations resulted in formation of aluminum adjuvant. A quantity of base needed for the aluminum adjuvant formation reaction to occur was provided by the lyophilized or spray dried formulations. pH of the final vaccine and median particle size d(0.5) from static light scattering measurements of some of the vaccines was measured. With complete reaction, the concentration of aluminum adjuvant formed for the lyophilized samples would be 266 mcg Al/mL.

### Formulation 23

10.49 mM aluminum chloride
9.44 mM sodium phosphate
In water, at acidic pH

### Formulation 24

6% sucrose
100 mM histidine
pH 7.6, lyophilized

### Formulation 25

6% sucrose
100 mM histidine
pH 7.0, lyophilized

### Formulation 26

6% sucrose
100 mM histidine
pH 6.0, lyophilized

### Formulation 27

1% sucrose
9% mannitol
50 mM phosphate
pH 7.4, spray dried

### Formulation 28

1% sucrose
9% mannitol
50 mM phosphate
pH 7.8, spray dried

TABLE 3
Median particle size of various formulations

| | Aluminum- and | | Median particle |
|---|---|---|---|
| Dried formulation to | Phosphate- containing | pH of aluminum | size d(0.5) |
| be reconstituted, | reconstitution solution | adjuvant | measured by |
| compring base or | (not an aluminum | suspension | static light |
| buffer | adjuvant suspension) | formed | scattering |
| Formulation 24 | Formulation 23 (0.94 mL) | 6.67 | 9.2 microns |
| (1 mL pre-lyo | | | |
| volume) | | | |
| Formulation 25 | Formulation 23 (0.94 mL) | 6.45 | 8.3 microns |
| (1 mL pre-lyo | | | |
| volume) | | | |
| Formulation 26 | Formulation 23 (0.94 mL) | 6.13 | 8.9 microns |
| (1 mL pre-lyo | | | |
| volume) | | | |
| Formulation 27 (100 | Formulation 23 | 6.36 | |
| mg spray dried | | | |
| powder) | | | |
| Formulation 28 (100 | Formulation 23 | 6.53 | |
| mg spray dried | | | |
| powder) | | | |

### Example 7

This example shows preparation of vaccine by co-packaging of lyobeads of an aluminum-containing solution, a phosphate/base-containing solution, and formulated antigen, and reconstitution with liquid to allow reaction and formation of aluminum adjuvant in the presence of antigen.

The following components were used to form the vaccine discussed in this example:

(1) Dried formulation comprising some of the reactants needed to form aluminum adjuvant (for example, an aluminum-containing compound)
(2) Dried formulation comprising others of the reactants needed to form aluminum adjuvant (for example, a phosphate-containing compound and/or a base)
(3) Dried formulation comprising antigen(s), any of 1-3 optionally comprising preservative(s), reconstituted with
(4) Liquid, comprising:
Water
Optionally buffer(s), salt(s), surfactant(s) and/or excipients
Optionally antigen(s)
Optionally additional adjuvant(s)
Optionally preservative(s)

### See FIG. 12.

### Embodiments in the Instant Example

(1) Before lyophilization, 92.66 mM aluminum chloride, 25% (w/v) trehalose
(2) Before lyophilization, 92.66 mM sodium phosphate tribasic, 25% (w/v) trehalose
(3) Before lyophilization, formulated antigens comprising:
320 mcg PnPs/mL CRM-PnPs (pneumococcal polysaccharide) conjugate antigen (polysaccharide type 19A)
100 mM histidine
20% (w/v) trehalose
50 mM sodium chloride
0.1% polysorbate 20
pH 7.5

Lyobeads of an aluminum-containing compound, of a phosphate/base containing-compound, and of an antigen formulation were prepared. A vaccine was made by co-packaging the three lyobead types and reconstituting with water. Reconstitution allowed reaction to form aluminum adjuvant in the presence of antigen.

Formulation 4 (for lyobeads comprising a monovalent conjugate vaccine (Type 19A shown below) antigen
20% (w/v) trehalose
100 mM histidine (base)
320 mcg PnPs/mL CRM-PnPS (pneumococcal polysaccharide) conjugate antigen (polysaccharide type 19A)
50 mM sodium chloride
0.1% polysorbate 20
   Formulation 7 (an aluminum containing compound)
92.66 mM aluminum chloride
25% (w/v) trehalose
   Formulation 8A (a phosphate/base containing compound)
92.66 mM sodium phosphate tribasic
25% (w/v) trehalose

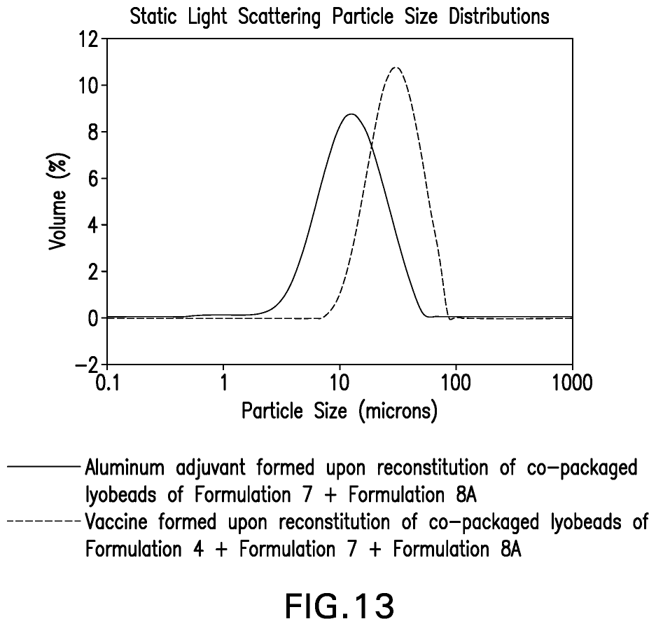

See the particle size analysis in FIG. 13. Vaccines made by co-packaging (in a vial or delivery device) lyobeads comprising an aluminum-containing compound, a phosphate/base-containing compound, and an antigen, which upon reconstitution allow the formation of the aluminum adjuvant in the presence of antigens, may be acceptable and efficacious.

### Example 8

This example shows preparation of a multivalent vaccine by reaction of aluminum and phosphate/base containing compounds to form aluminum adjuvant in the presence of antigens.

This example concerns a formulation made by filling two liquid solutions into a vial or small container, that result in the formation of aluminum adjuvant (optionally in the presence of antigen(s), salt(s), buffer(s), excipient(s), detergent(s), surfactant(s), preservative(s), stabilizer(s), dye(s), indicator(s)).

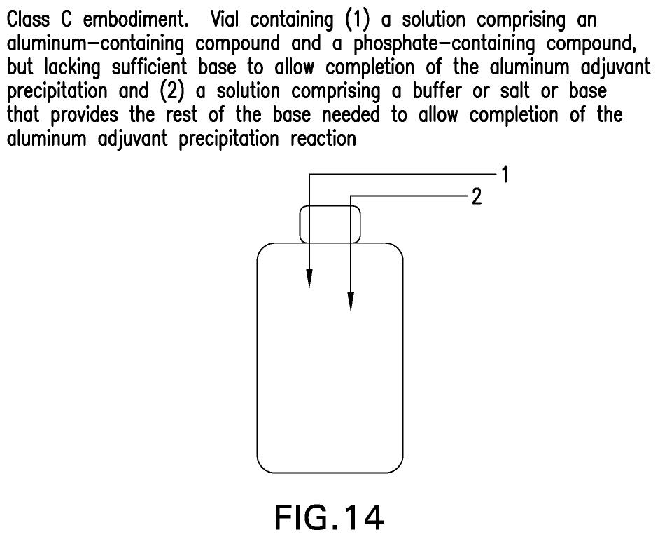

In one embodiment (see FIG. 14),

(1) a solution comprising an aluminum-containing compound and a phosphate-containing compound, but lacking sufficient base to allow completion of the aluminum adjuvant precipitation reaction is added to a vial, followed by an equal volume of

(2) a solution comprising a buffer or salt or base that provides the rest of the base needed to allow completion of the aluminum adjuvant precipitation reaction, and optionally comprising Antigen(s) and/or other components.

### Embodiment in the Instant Example

(1) 18.54 mM aluminum chloride (from aluminum chloride hexahydrate), 16.69 mM sodium phosphate, in water at acidic pH. (Prepared from mixture of equal volumes of 37.08 mM aluminum chloride and 33.38 mM sodium phosphate pH 7.)
(2) 15-valent PCV antigen formulation comprising:
128 mcg PnPs/mL CRM-PnPs (pneumococcal polysaccharide) conjugate antigen(s)
80 mM histidine, pH 7.0
6% sucrose
150 mM sodium chloride

ELISA-based relative potency and the fluorescence spectra of a multivalent vaccine were measured to demonstrate that the process of reacting aluminum chloride with sodium phosphate and sufficient base in the presence of antigens did not negatively impact the measured potency or fluorescence spectrum compared to antigens that had not experienced the adjuvant formation/precipitation reaction.

Herein, it was further demonstrated that an aluminum-containing compound and a phosphate-containing compound would not react to produce stable, precipitated aluminum adjuvant unless there was enough base present. Formulation 11 lacked the full amount of base necessary for the adjuvant formation reaction, and remains clear rather than forming a cloudy suspension of precipitated adjuvant solids; the lacking base is provided when mixed with Formulation 10 from the 80 mM histidine buffer. Thus, keeping only a portion of the base required for the reaction separate from the other components (in this example, the aluminum-containing compound and the phosphate-containing compound) can be sufficient to avoid reaction completion and formation of the aluminum adjuvant suspension, thus avoiding the possibility of freeze-thaw agglomeration of the adjuvant suspension in the time before the suspension is formed.

### Formulation 10

128 mcg PnPs/mL CRM-PnPS (pneumococcal polysaccharide) conjugate antigens (a blend of conjugates with 15 different polysaccharide types)
80 mM histidine, pH 7.0
6% sucrose
150 mM sodium chloride
In water

### Formulation 11

18.54 mM aluminum chloride hexahydrate
16.69 mM sodium phosphate
In water, at acidic pH

### Formulation 12

64 mcg PnPs/mL CRM-PnPS (pneumococcal polysaccharide) conjugate antigens (a blend of conjugates with 15 different polysaccharide types)
40 mM histidine
3% sucrose
100 mM sodium chloride
250 mcg Al/mL aluminum adjuvant
In water
pH 5.9

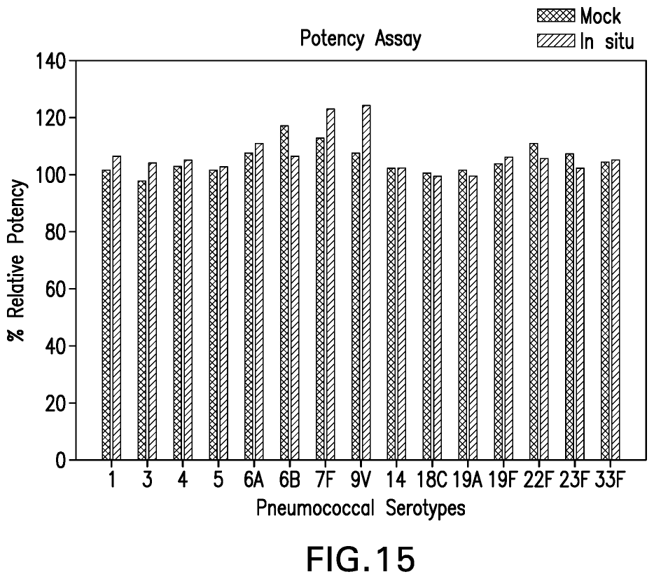

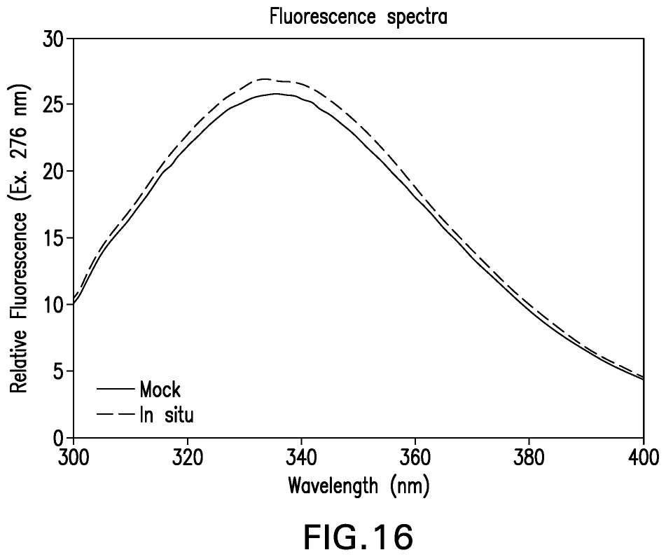

Equal volumes of Formulation 10 and Formulation 11 were mixed to result in Formulation 12. As a comparator, equal volumes of Formulation 10 and water were mixed, which did not result in the formation of aluminum adjuvant. The antigen potency for both samples was measured using an ELISA assay (FIG. 15), and the fluorescence spectra of both samples was measured (FIG. 16). By these assays, the structure and antigenicity of vaccine prepared by adjuvant precipitation in the presence of antigen appeared similar to the no aluminum adjuvant comparator (or “mock”) sample.

### Example 9

This example shows preparation of aluminum adjuvant by rapid, sequential addition of an aluminum-containing solution and a phosphate/base-containing solution on an automated vial filling line.

This example relates to a formulation formed by filling two liquid solutions into a vial using a filling line, that resulted in the formation of aluminum adjuvant (optionally in the presence of antigen(s), salt(s), buffer(s), excipient(s), detergent(s), surfactant(s), preservative(s), stabilizer(s), dye(s), indicator(s)), followed immediately by stoppering.

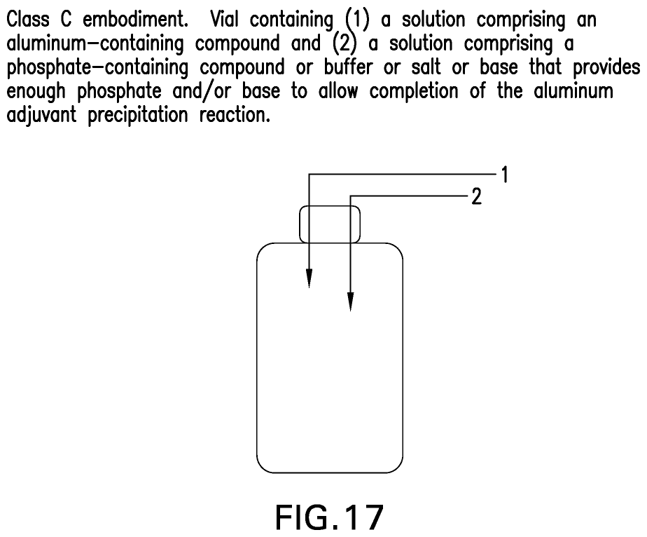

For example (see FIG. 17),

(1) a solution comprising an aluminum-containing compound is added, followed by an equal volume of;

(2) a solution comprising a phosphate-containing compound or buffer or salt or base that provides enough phosphate and/or base to allow completion of the aluminum adjuvant precipitation reaction, and optionally comprising antigen(s) and/or other components. Solutions may be added in reverse order, and volumes of addition could be designed intentionally to not be equal if desired.

### Embodiment in the Instant Example

(1) 18.53 mM aluminum chloride in water
(2) 100 mM sodium phosphate, in water, pH 8.1
(Alternate 2) 100 mM sodium phosphate, 9% (w/v) sucrose, in water, pH 8.1
(Another Alternate 2) 100 mM Tris*, 18.53 mM sodium phosphate, in water, pH 8.1
(*Tris is also known as tris(hydroxymethyl)aminomethane, and is also known by other names.)

A method for preparing aluminum adjuvant that involves adding multiple solutions to a vial or vaccine primary container with reaction and formation of the aluminum adjuvant in the vial was demonstrated. Compared to a typical aluminum adjuvant manufacturing approach, this approach has several advantages. It eliminates the need for other aluminum adjuvant manufacturing equipment and processes, and incorporates the process of aluminum adjuvant manufacture into the existing unit operation of vial (or primary container) filling. Solutions can be sterile filtered prior to aseptic fill and aluminum adjuvant formation. It also eliminates some manufacturing challenges associated with processing and filling pre-formed aluminum adjuvant suspensions. Solutions are easier to work with in manufacturing than suspensions, which can settle and become non-homogeneous.

A solution of aluminum-containing compound and each of three solutions of phosphate/base-containing compound (one per demonstration run, listed below) were added sequentially to 3-mL vaccine vials using two filling needles on a filling line, resulting in in-vial formation of aluminum adjuvant. The volumes of each solution dispensed were 0.5 mL per vial, for a final volume of 1 mL per vial. Vials were stoppered on the filling line. The speed of the filling line was set to approximately 53 vials per minute.

Formulation 13 (an aluminum-containing solution)
18.53 mM aluminum chloride
In water
   Formulation 14 (a phosphate/base-containing solution)
100 mM sodium phosphate
pH 8.1, in water
   Formulation 15 (a phosphate/base-containing solution)
100 mM sodium phosphate
9% (w/v) sucrose
pH 8.1, in water
   Formulation 16 (a phosphate/base-containing solution)
100 mM Tris (also known as tris(hydroxymethyl)aminomethane)
18.53 mM sodium phosphate
pH 8.1, in water

After addition of both components and complete precipitation reaction, the final concentration of aluminum adjuvant in the vials would be 250 mcg Al/mL. The pH of the final aluminum adjuvant suspensions was measured and is listed in the table below.

TABLE 4
pH measurements of various formulations

| (1:1 volume | pH | pH | Average |
|---|---|---|---|
| mixtures of) | measurement 1 | measurement 2 | pH |
| Formulation 13 + | 6.98 | 6.96 | 6.97 |
| Formulation 14 | | | |
| Formulation 13 + | 6.93 | 6.91 | 6.92 |
| Formulation 15 | | | |
| Formulation 13 + | 6.94 | 6.84 | 6.89 |
| Formulation 16 | | | |

The aluminum adjuvant had the expected visual appearance, and was very easy to resuspend by gentle inversion or agitation by hand, for at least 5.5 years, stored in stoppered vials at 2-8° C.

### Example 10

This example shows preparation of a multi-antigen aluminum adjuvanted vaccine by mixture of a solution of aluminum-containing compound with a second solution that comprised phosphate/base and antigens, resulting in reaction and formation of aluminum adjuvant in the presence of antigens and administration of this vaccine to subjects.

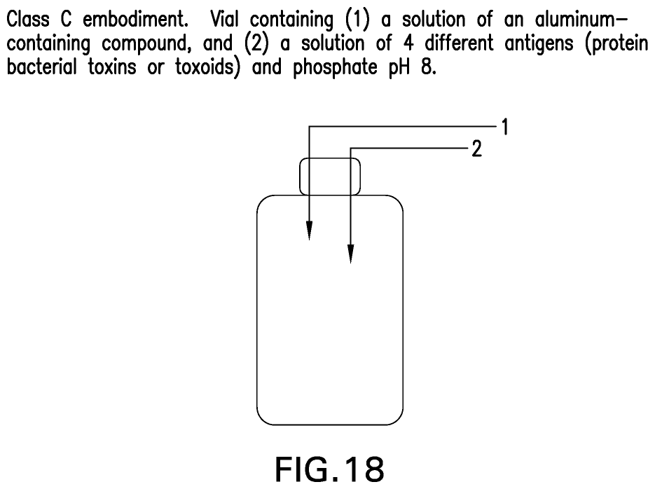

The present invention concerns a formulation formed by filling two liquid solutions into a vial or small container*, with (see FIG. 18)

(1) comprising a solution of an aluminum-containing compound, and
(2) comprising a solution of 4 different antigens (protein bacterial toxins or toxoids) and phosphate pH 8 as a buffer and source of base to allow completion of the aluminum adjuvant precipitation reaction in the presence of the antigens.
(*For example, using equal volumes such as 6 mL of each).

### Embodiment in the Instant Example

(1) 18.54 mM aluminum chloride, in water.
(2) 100 mM sodium phosphate, pH 8, 9% sucrose, in water, with 4 types of antigens:
Antigen 1 (20 mcg/mL vpi_5mTcdA, recombinant mutant toxin from C. difficile)
Antigen 2 (20 mcg/mL nap_5mTcdB, formaldehyde treated, recombinant toxoid from C. difficile)
Antigen 3 (5.6 mcg/mL 3mCdtA, recombinant mutant toxin subunit from C. difficile)
Antigen 4 (44.4 mcg/mL CdtB, toxin subunit from C. difficile)

A multi-antigen vaccine was prepared by combining aluminum chloride solution in water with multiple types of antigens formulated in aqueous sodium phosphate and sucrose, resulting in aluminum adjuvant formation in the presence of the antigens. The volume of vaccine prepared by this method was on the order of a multi-dose vaccine image for human or animal subjects. The vaccine was administered to subjects (hamsters) who received 4 doses over a course of several months. After receiving the vaccinations, group mean titers for two of the antigens present were compared between the antigen/aluminum-adjuvant co-precipitation vaccine and three other study groups that comprised antigens with three different forms of pre-formed aluminum adjuvant.

Formulation 13 (an aluminum-containing solution)
18.53 mM aluminum chloride
In water

### Formulation 29

Antigens related to C. difficile
   20 mcg/mL 5mTcdA
   20 mcg/mL 5mTcdB
   5.6 mcg/mL 3mCdtA
   44.4 mcg/mL CdtB
100 mM sodium phosphate, pH 8
9% sucrose
In water

Equal volumes (6 mL each) of Formulation 13 and Formulation 29 were mixed, resulting in aluminum adjuvant formation in the presence of antigens. Other vaccines tested in the hamster vaccination study included three additional vaccines which comprised antigens combined with three different pre-formed aluminum adjuvants. Immunogenicity (mean or geomean vaccination titers of groups, 8 hamsters per group) for the vaccines for two of the antigens present is reported in Table 5.

TABLE 5
Vaccine immunogenicity in hamster study.

| | Group titer to | Group titer to |
|---|---|---|
| Vaccine | antigen #1 | antigen #2 |
| Formulation 13 + Formulation 29 | 19,311 | 10,741 |
| (mixed 1:1 volumes) | | |
| Comparator vaccine with pre-formed | 16,958 | 4,867 |
| aluminum adjuvant #1 | | |
| Comparator vaccine with pre-formed | 27,352 | 10,672 |
| aluminum adjuvant #2 | | |
| Comparator vaccine with pre-formed | 31,677 | 11,705 |
| aluminum adjuvant #3 | | |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1-18. (canceled)

19. An article of manufacture comprising components
(1) an antigen, which is dried (i) or in solution (ii);
(2) a dried aluminum-containing and/or calcium-containing reagent; and
(3) a dried phosphate reagent and, optionally, a base;
wherein each of components (1), (2) and (3) are physically separated from the other components and co-packaged; and
wherein said aluminum-containing and/or calcium-containing reagent and said phosphate reagent and, optionally, the base, react when reconstituted with water to form an adjuvant.

20. The article of manufacture of claim 19, wherein component (1)(i), component (2), and component (3) are each provided as lyobeads.

21. The article of manufacture of claim 19, wherein component (1)(i), component (2), and component (3) are each provided as lyobeads and the article of manufacture comprises a first container comprising a mixture of the component (1)(i), component (2), and component (3) lyobeads, and optionally, a second container comprising water.

22-36. (canceled)

37. The article of manufacture of claim 19, wherein the antigen is a protein, a toxin or a toxoid, a polysaccharide, or one or more pneumococcal polysaccharide-protein conjugates.

38. The article of manufacture of claim 37, wherein the polysaccharide or pneumococcal polysaccharide is a S. pneumoniae polysaccharide or S. pneumoniae pneumococcal polysaccharide.

39. The article of manufacture of claim 37, wherein the protein or protein conjugate is a CRM197 protein or a CRM197 protein conjugate, a diphtheria toxoid (DT) protein or a DT protein conjugate, or a tetanus toxoid (TT) protein or a TT protein conjugate.

40. The article of manufacture of claim 37, wherein the protein or protein conjugate is a CRM197 protein or a CRM197 protein conjugate.
